(19) United States
(12) Patent Application Publication
(10) Pub. No.: US 2012/0021440 A1
(43) Pub. Date: Jan. 26, 2012

(54) METHODS FOR TESTING LIGAND BINDING TO G PROTEIN-COUPLED RECEPTORS

(75) Inventors: Jeffrey Horton, Cardiff (GB); Peter Tatnell, Cardiff (GB)

(73) Assignee: GE HEALTHCARE UK LIMITED, LITTLE CHALFONT (GB)

(21) Appl. No.: 13/262,129

(22) PCT Filed: Mar. 26, 2010
(86) PCT No.: PCT/EP2010/053984

§ 371 Date: Sep. 29, 2011

(30) Foreign Application Priority Data
Mar. 30, 2009 (GB) 0905419.8

(51) Int. Cl. G01N 21/78; C12N 5/10; C12N 15/85; C12N 9/96; C12N 15/62

(52) U.S. Cl. 435/7.21; 435/188; 536/23.2; 435/320.1; 435/325

(57) ABSTRACT

The present invention relates to methods for testing for the binding of a ligand to a G Protein-Coupled Receptor. In particular, the methods of the invention are useful in high throughput screening for ligands which bind to G Protein-Coupled Receptors.

### CROSS-REFERENCE TO RELATED APPLICATIONS

This application is a filing under 35 U.S.C. §371 and claims priority to international patent application number PCT/EP2010/053984 filed Mar. 26, 2010, published on Oct. 7, 2010 as WO 2010/112417, which claims priority to application number 0905419.8 filed in Great Britain on Mar. 30, 2009.

### FIELD OF THE INVENTION

The present invention relates to the field of cell biology, molecular biology and drug screening. In particular, the invention relates to G Protein-Coupled Receptors (GPCRs) and to methods for testing the binding of ligands to GPCRs.

### BACKGROUND OF THE INVENTION

### G Protein-Coupled Receptors

G protein-coupled receptors (GPCRs), also known as seven transmembrane domain receptors, 7™ receptors and G protein-linked receptors (GPLR), comprise a large protein family of trans-membrane receptors that bind molecules outside the cell and activate signal transduction pathways and, ultimately, cellular responses. GPCRs are found only in eukaryotes, including yeast, plants, flagellate protozoa, animals. The ligands that bind and activate these receptors include light-sensitive compounds, odours, pheromones, hormones, neurotransmitters, and drugs, and vary in size from small molecules such as peptides to large proteins. As GPCRs are involved in many diseases, they are the target of many modern medicines. Drugs active at GPCRs have therapeutic benefit across a broad spectrum of human diseases as diverse as pain, cognitive dysfunction, hypertension, peptic ulcers, rhinitis, asthma, inflammation, obesity and cancer, as well as cardiovascular, metabolic, gastrointestinal, visual and neurodegenerative diseases. Of the total clinically marketed drugs, greater than 30% are modulators of GPCR function, representing 9% of global pharmaceutical sales, making GPCRs the most successful of any target class in terms of drug discovery. GPCRs represent the single most important class of drug targets and significant targets in drug discovery. Indeed, 20% of the top fifty best selling drugs act at GPCRs, which equates to approximately $25 billion in terms of pharmaceutical sales per annum. However, current drugs exhibit their activity at less than 10% of known GPCRs, implying that there is large potential for further discovery. Indeed, the DNA sequences of a large number of GPCRs can be found in public databases, among other sources, leading to identification of putative so-called “orphan receptors”. These orphan receptors are defined as those not acted upon by a known endogenous ligand. One challenge for the drug development industry is to associate the many orphan GPCRs with disease to potentially identify novel pharmaceutical agents of the future.

GPCRs are closely associated with heterotrimeric G-proteins that are bound to the inner face of the plasma membrane. G-proteins are key molecular components in the intracellular signal transduction following ligand binding to the extracellular domain of a GPCR. The G-protein subunits historically are designated α, β, and γ, and their classification is largely based on the identity of their distinct α subunits, and the nature of the subsequent transduction event (Table 1). Further classification of G-proteins has come from cDNA sequence homology analysis. G-proteins bind either guanosine diphosphate (GDP) or guanosine triphosphate (GTP), and possess highly homologous guanine nucleotide binding domains and distinct domains for interactions with receptors and effectors.

When the GPCR “system” is inactive (i.e. in the absence of ligand), GDP is bound to the Gα subunit. An agonist-receptor complex induces conformational changes in the GPCR/G-protein complex, which facilitates preferential binding of GTP to the Gα subunit, in part by promoting the dissociation of bound GDP. This so-called “guanyl nucleotide exchange” is critical. Binding of GTP activates the Gα subunit, leading to dissociation through space from the Gβγ dimer. The Gα and Gβγ subunits are then able to subsequently activate, either independently or in parallel, downstream effectors such as adenylate cylase, calcium, phospholipase activity or other ions.

Termination of signal transduction results from hydrolysis of bound GTP to GDP by a GTPase enzyme that is intrinsic to the α subunit, leading to re-association of α and βγ subunits. Thus, G-proteins serve as regulated molecular switches capable of eliciting bifurcating signals through α and βγ subunit effects. The switch is turned on by the receptor and it turns itself off within a few seconds, a time sufficient for considerable amplification of signal transduction.

TABLE 1
Examples of the relationship of G Protein-Coupled
Receptors and Signalling Pathways

| G-Protein Subunit | |
|---|---|
| Regulation | Effectors/Signalling Pathways |
| αs | Adenylate cyclase (cAMP) ↑ |
| αi | Adenylate cyclase (cAMP) ↓ |
| αo | Ca²⁺ ↓ |
| αq | Phospholipase C (IP₃) ↑ |
| α13 | Na⁺/H⁺ exchange ↑ |
| αt | cGMP-phosphodiesterase (vision) ↑ |
| αolf | Adenylate cyclase (cAMP) ↑ |
| βγ | K⁺ channels |
| βγ | Adenylate cyclase (cAMP) ↑ or ↓ |
| βγ | Phospholipase C (IP₃) |

### Structure of GPCRs

GPCRs are integral hydrophobic membrane proteins that span the plasma membrane in seven α-helical segments. The extracellular binding site for small GPCR-active ligands is a pocket within the bundle of membrane-spanning helices, but a substantial extracellular domain is important for the binding of the negatively charged ligands. GPCRs are activated by an external signal in the form of a ligand or other signal mediator. This creates a conformational change in the receptor, causing activation of a G-protein. Further effect depends on the type of G-protein. The receptors interact with G proteins at their cytoplasmic face, and it has been possible to define specific regions within GPCR structures that are responsible for regulation of and selectivity among the different G-proteins.

In order to study GPCR activation, a variety of functional biochemical and cellular assay methodologies are typically used. Examples of functional assay systems for monitoring GPCR activation include the intracellular measurement of the GPCR effector targets, cAMP, cGMP and IP₃. A number of homogeneous assay methodologies such as Scintillation Proximity Assay (SPA), Fluorescence Polarization (FP) and Enzyme Fragment Complementation (EFC) have been successfully used for the measurement of these agents. Furthermore, as described above, ligand-induced stimulation of GPCRs results in the exchange of GDP for GTP, and this event can be monitored by the binding of radiolabelled [³⁵S] GTPγS, for example.

In the process of drug discovery and lead optimisation, there is a requirement for faster, more effective, less expensive and especially information-rich screening assays that provide simultaneous information on various compound characteristics and their affects on various cellular pathways (i.e. efficacy, specificity, toxicity and drug metabolism). Such assays would allow drug discovery to identify drug candidates capable of activating or blocking GPCR signalling. Thus, for drug discovery, there is a need to quickly and inexpensively screen large numbers of chemical compounds to identify new drug candidates, including receptor agonists, inverse agonists and antagonists as well as inhibitors of GPCRs and GPCR-dependent pathways. These chemical compounds may be collected in large libraries, sometimes exceeding one million distinct compounds.

Traditional biochemical approaches for assaying GPCRs have relied upon measurements of ligand binding, for example with filter binding assays (heterogeneous) or with SPA (homogeneous). Although such assays are inexpensive to carry out, development time can be lengthy in some cases. A major problem is that the development of a traditional assay requires specific reagents for every target of interest including purified protein for the target against which the screen is to be run. Often it is difficult to express the protein of interest and/or to obtain a sufficient quantity of the protein in pure form.

Although binding assays are the gold standard for pharmacology and studies of structure-activity relationships (SAR), it is not usually possible to perform target validation with binding assays. The increased numbers of drug targets identified by genomic approaches has driven the development of gene-to-screen approaches to interrogate poorly-defined targets, many of which rely on cellular assay systems. Speculative targets are most easily screened in a format in which the target is expressed and regulated in the biological context of a cell, in which all of the necessary components are pre-assembled and regulated. Cell-based assays are also critical for assessing the mechanism of action of new biological targets and biological activity of chemical compounds. In particular, there is a need to “de-orphanise” those GPCRs for which natural activating ligand has not been identified. Various approaches to “de-orphanisation” have been adopted including array-screening against families of known ligands. Current cell-based assays for GPCRs include measures of pathway activation (Ca²⁺ release, cAMP generation or transcriptional activity); measurements of protein trafficking by tagging GPCRs and down stream elements with GFP; and direct measures of interactions between proteins using Föorster resonance energy transfer (FRET), bioluminescence resonance energy transfer (BRET) or yeast two-hybrid approaches.

### DEFINITIONS

The meaning of the terms “agonist”, “inverse agonist” and “antagonist” as used herein are based on the definition given in: “Definitions, GlaxoWellcome Pharmacology Guide”, which can be found online.

An “agonist” is a compound or drug which binds to a receptor and activates it, producing a pharmacological response (e.g. contraction, relaxation, secretion, enzyme activation, etc.).

An “inverse agonist” is a compound or drug which produces an effect opposite to that of an agonist, yet acts at the same receptor. The best established examples act at the benzodiazepine receptor. Such compounds have also been described as “negative antagonists”, or as having “negative efficacy”.

An “antagonist” is a compound or drug which attenuates the effect of an agonist. It may be competitive, i.e. it binds reversibly to a region of the receptor in common with an agonist but occupies the site without activating the effector mechanism. The effects of a competitive antagonist may be overcome by increasing the concentration of agonist, thereby shifting the equilibrium and increasing the proportion of receptors which the agonist occupies.

Alternatively, antagonists may be non-competitive, where no amount of agonist can completely overcome the inhibition once it has been established. Non-competitive antagonists may bind covalently to the agonist binding site (“competitive irreversible antagonists”), in which case there is a period before the covalent bond forms during which competing ligands can prevent the inhibition. Other types of non-competitive antagonists act allosterically at a different site on the receptor or an associated ion channel.

The term “testing” as used herein, shall include but not be limited to, detecting, measuring and/or quantifying.

“Binding” is defined herein as an event which involves an agent or molecule selectively interacting with one or more sites on another molecule.

A “ligand” as used herein, shall mean a substance or compound that is able to bind to form a complex with a biomolecule to serve a biological purpose such as triggering a biological response.

An “homogeneous assay is defined herein as a method that does not involve a wash step, for example a step without physical separation.

“Optical signal”, as used herein, shall mean light emission of any wavelength. For the avoidance of doubt, this includes luminescence, fluorescence and any form of electro magnetic radiation such as x-rays.

“Fluid sample” shall mean a liquid solution or a suspension.

### Cell Based Assays

Traditional cell-based assays for GPCRs often rely upon measurements of intracellular calcium flux. Calcium release from intracellular stores is stimulated by specific classes of GPCRs upon their activation; in particular those GPCRs that couple to a G-protein known as Gq (or Gq/11). Fluorescent and luminescent assays of calcium release have been generated by loading cells with dyes that act as calcium indicators. Fluorescent calcium indicators such as Fura-2, Indole-1, Fluo-3 and calcium green have been widely used for measurement of intracellular calcium measurement. Such indicators and associated instrumentation such as FLIPR (Molecular Devices, Sunnyvale, Calif., USA) are well established tools. Luminescent assays of calcium transients can be also carried out, by the introduction of aequorin into cells, usually with genetic engineering techniques. Aequorin emits blue light in the presence of calcium, and the rate of photon emission is proportional to the free calcium concentration within a specific range. Cells expressing the GPCR of interest are loaded first with coelenterazine to activate the aequorin, and then the compounds to be tested are added to the cells and the results measured with a luminometer. To extend these assays to non-Gq-coupled receptors, various engineering strategies have been used, including the use of a promiscuous Gα protein construct such as Gα16 that is capable of coupling a wide range of GPCRs to phospholipase C(PLC) activity and calcium mobilisation.

Adenylyl cyclases are a family of membrane-bound enzymes that are linked to G protein-coupled receptors and influence the regulation of cell function in virtually all cells. cAMP is synthesized by adenylyl cyclase in response to activation of many receptors; stimulation is mediated by Gs, and inhibition by one or more closely related G proteins termed Gi's. There exist at least ten tissue-specific adenylyl cyclases each with its unique pattern of regulatory responses. Several adenylyl cyclase isozymes are inhibited by the G protein βγ subunits, which allow activation of G proteins other than Gs to inhibit cyclase activity. Other isozymes are stimulated by Gβγ subunits, but this stimulation is dependent upon concurrent stimulation by the α subunit of Gs. Still other isozymes are stimulated by Ca²⁺ or Ca²⁺⁻-calmodulin complexes. Finally, each of the isozymes has its own pattern of enhancement or attenuation by phosphorylation or other regulatory influences, providing a broad array of regulatory features to the target cells where these isoforms are expressed.

cAMP is a ubiquitous second messenger and functions as one of many signalling molecules enabling cells to respond to external signals. cAMP assays are used to monitor cellular responses to either Gs or Gi-coupled receptor activation. Typically, with cAMP, the binding of a hormone, agonist or neuromodulator to its receptor is followed by activation or inhibition of a G-protein which, in turn, activates adenylate cyclase, evoking the generation of cAMP from ATP. The activation of protein kinase A by cAMP results in the phosphorylation of specific substrates, which include enzymes, ion channels and transcription factors. Because cAMP can activate a cascade of reactions, the involvement of cAMP greatly amplifies the cellular response to a variety of drugs and hormonal stimuli. Therefore, measurement of intracellular cAMP generation has become an established means of screening for antagonists and agonists of receptors linked to adenylate cyclase via either inhibitory or stimulatory G-proteins.

Fluorescent dyes, and fluorescent proteins such as Green Fluorescent Protein (GFP), Yellow Fluorescent Protein (YFP), Blue Fluorescent Protein (BFP) and Cyan Fluorescent Protein (CFP), have also been used as cellular sensors of cAMP and calcium. The first protein indicator for cAMP consisted of the cyclic AMP-dependent protein kinase, PKA, in which the catalytic and regulatory subunits were labelled with fluorescein and rhodamine, respectively, so that cAMP-induced dissociation of the subunits disrupted FRET between the dyes. Replacement of the dyes by GFP and BFP made this system genetically encodable and eliminated the need for in vitro dye conjugation and microinjection.

Transcriptional reporter assays provide a measurement of pathway activation or inhibition in response to an agonist/antagonist, and have been used extensively in GPCR studies. Reporter-gene assays couple the biological activity of a receptor to the expression of a readily detectable enzyme or protein reporter. Naturally-occurring gene promoters or synthetic repeats of a particular transcription factor response element can be inserted upstream of the reporter gene to regulate its expression in response to signalling molecules generated by activation of a specific pathway in a live cell. Such drug screening systems have been developed with a variety of enzymatic and fluorescent reporters, including β-galactosidase, luciferase, alkaline phosphatase, GFP, β-lactamase and others. Transcription reporter assays are highly sensitive screening tools; however, they do not provide information on the mechanism of action of the compound. The latter would enable mapping of the components of the pathway, leading to transcription, or enable studies of the individual steps within signalling cascades.

Luminescence as a detection method has gained in popularity in recent years because these assays can deliver 10 to 1000 fold higher assay sensitivity than assays using fluorescent proteins (Wood, 2007, Progeag Notes, Promega Corporation). This greatly increased sensitivity can substantially improve assay performance when applied to complex biological samples. The reason for the improved performance is the high luminescence signal compared with background, as opposed to assay results typically obtained with fluorescent proteins.

High content screening (HCS) is an approach that, in one format, relies upon imaging of cells to detect the sub-cellular location and trafficking of proteins in response to stimuli or inhibitors of cellular processes.

Kimple et al. (Combin. Chem. & High Through. Screen., 2003, 6, (4) 399-407) describe established and emerging fluorescence-based assays for G-protein function.

Fluorescent probes can be used in HCS. For example, GTP has been labelled with the fluorescent dye, BODIPY®, and used to study the on/off-rates of GTP hydrolysis by G-proteins. Fluorescein-labelled myristoylated Gαi has also been used as a ligand that binds Gβγ in order to study the association and dissociation of G-protein subunits.

GFP has been used to analyse key signalling events within cells. By fusing in-frame a cDNA for GFP to a cDNA coding for a protein of interest, it is possible to examine the function and fate of the resulting chimera in living cells. This strategy has now been applied to nearly all known elements of G-protein coupled pathways including the receptors themselves, G-protein subunits such as Gα; β-arrestin, RGS proteins, protein kinase C and other intracellular components of G-protein-coupled pathways. For example, GPCRs have been tagged with GFP in order to monitor receptor internalization. A fusion protein comprising GFP-β-arrestin has been shown to co-localise with thyrotropin-releasing hormone receptor 1 in response to agonist. GFP has been introduced internally to G-proteins, creating a Gα/GFP chimera, which has been shown to translocate to the cell membrane upon GPCR activation. GFP tagging has also been used to monitor intracellular signalling events. GFP tagged RGS proteins were selectively recruited to the plasma membrane by G-proteins and their receptors. GFP-tagged protein kinase C(PKC), which is activated by the release of diacylglycerol from cell membranes, has been used to monitor translocation of the kinase in response to cell signalling. In addition, GFP-tagged connexion has been used to monitor intracellular calcium flux.

US20050181452 (Westwick, J. et al.) describes a number of assays for GPCRs and their signalling pathways. US20050181452 discloses the use of a high content fluorescent-based cellular assay for GPCRs involving YFP complementation fragments coupled to the GPCR Frizzled 4 and the RGS-protein RGS2. On activation of the GPCR e.g. in response to the binding of the Frizzled 4 ligand wnt, a fluorescent YFP signal is generated which can be captured by microscopy.

The document also describes a fluorescent FRET-based assay involving the generation of two fusion proteins in which i) CFP is coupled to Gαi1 and ii) YFP is coupled to RGS4. The interaction between the Gαi1 and RGS4 moieties on GPCR activation is monitored by the induction of a FRET signal generated by the transfer of energy from the two fluorescent protein fusion partners. The two fluorescent proteins are brought into close contact via the interaction between Gαi1 and RGS4.

U.S. Pat. No. 5,891,646 and US6110693 (Norak Bioscience) describe GFP-tagged β-arrestin and this has been used to monitor GPCR activation by imaging the subcellular redistribution of β-arrestin in response to agonist. However, the signal generated from GFP translocation methods is very low and is prone to instrument interference which can result in poor assay results

WO 2005/121755 (Commonwealth Sci.) and Leifert et al. (2006) Anal. Biochem., 355, 201-212 describe a cell-free GPCR and ligand assay using chemically-generated fluor molecules in a FRET assay.

An homogeneous GTP binding assay for GPCRs based on time-resolved FRET has previously been described (Frang, H., et al., GTP binding assay for GPCRs based on TR-FRET, Poster PO 8123, Ninth Annual Society for Biomolecular Screening, Portland, Oreg., 21-25 Sep. 2003). In this assay, a biotinylated BIOKEY® peptide is employed that recognizes only the GTP bound form of the Gα subunit. The biotinylated peptide enables binding of streptavidin-europium in close proximity to an acceptor-labelled GTP, which is also bound to the Gα subunit. FRET occurs as a result of interaction between the streptavidin-europium (donor) and the fluorescently-labelled GTP analogue (Alexa647-GTP).

WO2004/035614 (Karo Bio AB) discloses non-naturally occurring Gα conformation specific peptides that bind to the alpha subunit of G-proteins. The document discloses the use of these peptides in monitoring the activation and deactivation of GPCRs.

WO 2006/035207 (GE Healthcare UK Limited) describes fluorescent cyanine dye labeled nucleotide analogues in which the cyanine dye is coupled to the γ-phosphate group of a nucleoside triphosphate. These GTPase resistant analogues can be used in an homogenous FRET-based assay to measure the binding of guanine nucleotides to GPCR polypeptides, or alternatively, to measure the effect of an exogenous ligand on GPCR binding. Further uses of similar GTPase resistant GTP analogues in GPCR binding assays are disclosed in WO2006/035208 (GE Healthcare UK Limited).

While a variety of fluorescent dye-nucleotide conjugates are available, the selection of a particular fluorescent label for use in a protein binding assay can be problematic, since the electronic and spatial requirements of the binding site of the protein of interest are difficult to predict a priori. There is therefore, still a requirement for new GTP analogues that may be used for quantitating G-proteins and for studying the kinetics of agonist induced guanine nucleotide exchange in in vitro assays and in cellular systems.

### Enzyme Complementation

β-galactosidase (also referred to as “β-gal” or “β-gal” in the scientific literature) is a tetrameric enzyme with a MW of 464,000. Each identical subunit contains 1021 amino acids, encoded in Escherichia coli (E. coli) by the lacZ gene of the lac operon promoter. In E. coli, intracistronic β-galactosidase complementation is a naturally occurring phenomenon, and involves α and ω complementation of amino and carboxyl-terminal domains of the lacZ gene product.

Ullmann et al. (Ullmann A, Perrin D, Jacob F, and Monod J (1965). J Mol. Biol., 12, 918-923) described the complementation of β-galactosidase in E. coli. A peptide was found (Peptide “w”) that was present in extracts of various mutants (“ω donors”) of the lacZ gene. The ω peptide complemented β-galactosidase activity when added to extracts containing a β-galactosidase (−ve) mutant (“ω acceptor”). The ω enzyme acceptor peptide (EA) has since been found to lack residues 11-41, and is frequently referred to as the M15 protein, since it is a product of the

lacZ M15 allele. Sucrose density assessments suggested a MW of 30,000 to 40,000 for the ω peptide and in an operator-distal segment of the lacZ gene. A following publication by Ullmann et al. (Ullmann A, Jacob F, and Monod J, 1967 J Mol. Biol., 24, 339-343) described how extracts from various β-galactosidase-negative mutants were screened for their capacity to complement with extracts of partial deletions of the operator-proximal segment (“α”) of the lacZ gene. Together, the operator-distal (ω) and the operator-proximal (α) part of the lacZ gene account for about one-half of both the structural length and Mᵣ of the lacZ gene product for β-galactosidase.

Zamenhoff and Villarejo (Zamenhof P, Villarejo M, 1972, J. Bacteriol., 110, 171-178) demonstrated in vivo α-complementation of β-galactosidase in 16 lacZ gene terminator (nonsense) mutant strains of E. coli upon introduction of a gene fragment specifying production of a mutant lacZ polypeptide containing a small deletion in the N-terminal region of the enzyme monomer.

Since then, many sequence variants of donor and acceptor species of β-galactosidase have been described, reviewed by Eglen (Eglen R, 2002, Assay and Drug Development Technologies, 5, 97-105; DiscoveRx). In particular, a variation developed by DiscoveRx is a system for complementation of a small 4 kDa a fragment enzyme donor (ED) peptide (termed “ProLabel”) with an ω deletion mutant of the enzyme acceptor (EA). Further work reviewed by Olson and Eglen (Olson K & Eglen R, 2007, Assay and Drug Development Technologies, 5, 137-144) describes the 47-mer enzyme donor (ED) sequence.

In addition to enzyme complementation of β-Galactosidase, complementation is a common phenomenon now reported for other proteins, including dihydrofolate reductase (Remy I & Michnick S, 2001, PNAS, 98, 7678-7683), β-lactamase (Wehrman T et al., 2002. PNAS, 99, 3469-3474), luciferase (Ozawa T., et al., 2001. Anal Chem., 73, 2516-2521), ubiquitinase (Rojo-Niersbach E et al., 2000, Biochem J., 348, 585-590), alkaline phosphatase (Garen A. & Garen S., 1963, J. Mol. Biol. 7, 13-22) and tryptophan synthase (Yanofsky, C., & Crawford, I. P., 1972, Enzymes 7, 1-31).

Henderson et al., (1986) Clinical Chemistry, 32(9), 1637-1641 (Microgenics) describes genetic engineering of β-galactosidase which led to the development of a homogeneous immunoassay system.

U.S. Pat. No. 5,120,653 (Microgenics) describes a vector comprising a DNA sequence coding for an enzyme-donor polypeptide.

U.S. Pat. No. 5,643,734 and U.S. Pat. No. 5,604,091 (Microgenics) describe methods and compositions for enzyme complementation assays for qualitative and quantitative measurement of an analyte in a sample.

WO 2003/021265 (DiscoveRx) describes a genetic construct intracellular monitoring system provided for producing biologically active fusion proteins comprising a sequence encoding an enzyme donor (“ED”) sequence fused in reading frame to a sequence encoding a surrogate of a mammalian protein of interest, where the fusion protein has the function of the natural protein. Furthermore, a vector is described comprising a transcriptional and translational regulatory region functional in a mammalian host cell, a sequence encoding the ED joined to a multiple cloning site, an enzyme acceptor (“EA”) protein or enzyme acceptor sequence encoding such protein that is complemented by the ED to form a functional enzyme such as β-galactosidase. Mammalian cells are employed that are modified to provide specific functions.

U.S. Pat. No. 7,135,325 (DiscoveRx) describes short enzyme donor fragments of β-galactosidase of not more than 40 amino acids.

WO 2006/004936 (DiscoveRx) describes methods for determining the intracellular state of a protein as well as modifications to the protein. The method involves introducing a surrogate fusion protein comprising a member of an enzyme fragment complementation complex and a target protein. After exposing cells transformed with the surrogate fusion protein to a change in environment (e.g. a candidate drug), the cells are lysed, the lysate separated into fractions or bands by gel electrophoresis and the proteins transferred by Western blot to a membrane. The bands on the membrane are developed using the other member of the enzyme fragment complementation complex and a substrate providing a detectable signal.

US2007/0105160 (DiscoveRx) describes methods and compositions for determining intracellular translocation of proteins employing β-galactosidase fragments that independently complex to form an active enzyme. Engineered cells have two fusion constructs: one fragment bound to a protein of interest; and the other fragment bound to a compartment localizing signal. The cells are used to screen compounds for their effect on translocation, where a substrate containing high ionic strength solution is used for detection of the enzyme complex.

### Tagging the N-Terminus of Gβ and Gγ Subunits

At present 16 genes are known to encode for Human Gα subunits, 5 for Gβ and 12 genes for Gγ. Classically, G proteins are divided into four families based upon their Gα subunits—Gαi, Gαs, Gαq/11 and Gα12/13.

Both the Gβ and Gγ subunits have been N-terminally tagged with Enhanced Cyan Fluorescent Protein, ECFP (Bunnemann et al., 2003, PNAS, 100 16077-16082). In these experiments the authors demonstrated a functional signaling pathway from receptors to heterotrimeric G proteins to downstream signaling events. The ECFP-tagged proteins were functionally equivalent to wild-type proteins.

This study demonstrates that the N-terminals of both Gβ and Gγ subunits can be fused to reporter molecules (e.g. ECFP or β-Galactosidase donor peptide) without affecting heterotrimeric G-protein function.

### Gα Subunits

Gα subunits contain two domains: a GTPase domain involved in the binding and hydrolysis of GTP and a helical domain that buries GTP within the protein core. The helical domain is the most divergent domain amongst the Gα families and contains 3 flexible switch regions designated I, II and III. These become rigid and “structurally well ordered” in the GTP-bound state.

When Gα is bound to GDP a hydrophobic pocket is created that is crucial for Gβγ dimer binding. On GTP binding a conformational change occurs that removes the pocket and reduces the Gα affinity for Gβγ.

The Gα N-terminal region is “structurally well ordered” and is extremely important for the Gα interaction with Gβ. The extreme C-terminal region (in particular the last 5 residues) of Gα is an important mediator of GPCR and G protein interactions e.g. antibodies recognising the Gα C-terminal blocks GPCR/G-protein signalling. Therefore any alteration to either the N or C-terminal regions of Gα proteins has a dramatic effect on GPCR/G-protein signalling.

Recently, the cDNA encoding EYFP (and Renilla luciferase) has been inserted into the internal AB loop of the helical domain of Gαi. These cDNAs encode peptides that are ˜240 and 315 residues respectively (Bunnemann et al 2003 PNAS 26, 16077-16082).

Similar constructs have also been generated for Gαs and Gαq (see Yu et al., (2002) Mol Pharm, 61, 352-359 and Hughes et al. (2001), JBC 276, 4227-4235, respectively). The resulting recombinant fusion proteins were functionally identical to the native Gα proteins in terms of GPCR coupling and signal transduction

Structural models of Gα1 have been described showing the location of the AB loop in the helical domain into which the cDNA encoding EYFP was inserted (Bunemann et al. 2003, PNAS, 100 16077-16082). EYFP was inserted between residues ˜MGRL-linker-EYFP-linker-KIDF—these correspond to residues L90 and K91 of human Gα1. Similar constructs have also been generated for Gαs and Gαq.

### Regulators of G-Protein Signaling

Regulators of G-protein signalling (RGS) are a family of GTPase-activating proteins (GAP). These all contain the RGS box that accelerates the intrinsic GTPase activity of the Gα subunit thereby promoting heterotrimeric G-protein re-association and termination of signal transduction. Several RGS proteins also contain additional functional domains/motifs that extend their roles e.g. co-ordinating heterotrimeric G-protein and tyrosine kinase signalling pathways etc.

### RGS4

RGS4 functions as a GAP protein and has been shown to bind Gαi1 (and Gαq). In this instance structural studies have shown that RGS4 exclusively interacts with the three switch domains of Gαi1. These Gαi1 switch regions are composed of residues 179-185, 204-213 and 235-237 (switch 1, switch 2 and switch 3 respectively). These residues are intimately associated with the binding and hydrolysis of GTP and interact with the most highly conserved region in RGS4 (i.e. RGS4 residues 83-88, 124-134 and 159-167 with switch regions 1/2, 2/3 and 1 respectively). No significant interaction occurs between RGS4 and the Gαi1 AB loop region (Kimple et al., 2002 Nature 416 878-881). Interestingly RGS4 binding also blocks the phospholipase C interaction site therefore RGS4 acts as both an antagonist of effector binding and as a GAP.

The RGS4-Gαi1 complex is shown schematically in FIG. 1. RGS4 (10) does not make significant contact with the α-helical domain of Gαi1 (20) and interacts almost exclusively with the switch regions of the Ras-like domain (Tesmer et al. 1997, Cell 89 251-261). The Gαi1 N terminus (22), C terminus (24) and the Gαi1 switch region (26) of Gαi1 (20) are shown in the figure.

When expressed in HEK293 cells an EGFP-RGS4 fusion protein localises to the cytoplasm. However on the co-expression of either constitutively activated Gαi or M2 muscarinic receptor the fusion protein adopts a plasma membrane localisation. In Sf9 cells expressing the receptor plus Gαi, EGFP-RGS4 was demonstrated to function in a similar fashion to wild-type RGS4 and act as a GTPase-activating protein (see Roy et al., 2003 Mol. Pharm. 64, 587-593).

Similar results are observed when an EGFP-RGS2 fusion protein is co-expressed with either the beta 2 adrenergic receptor or the angiotensin II type 1A receptor. These receptors activate Gαs and Gαq respectively.

Other candidate RGS proteins include, RGS3 this bind to A1F4⁻ activated Gα11 (Gαq) upon stimulation of HEK293 cells with endothelin-1 and RGS-PX1 which binds Gαs via its RGS domain in HEK293 cells expressing the beta 2 adrenergic receptor on stimulation with isoproterenol. In both cases the E. coli β-galactosidase N-terminal amino acids residues 3-92 can be fused to the N-terminal of each protein. Constructs similar to those described above for Gαi containing internal EGFP and Renilla luciferase sequences have also been generated for Gαs and Gαq.

### The GoLoco Motif

RGS proteins such as RGS12 and RGS14 contain the GoLoco motif at their C-terminal end. This motif (110) has been shown to bind to the GDP-bound Gαi (120) (adenylate cyclase inhibitory) forming a Gα-GDP/GoLoco complex (130) preventing both GDP release and the re-association of Gα with Gβγ (140), thus permitting continued Gβγ effector interactions (150) in the absence of Gα activation (FIG. 2).

With regards to the RGS14 GoLoco-Gαi1 complex, the GoLoco peptide interacts with residues in both the GTPase and helical domains of Gαi1. However no specific interaction occurs within the AB loop region of Gαi1.

The crystal structure of the RGS14 GoLoco region complexed to the GDP-bound Gαi1 has been solved. This 36 amino acid peptide exhibits specific selectivity towards Gαi1, Gαi2 and Gαi3 but not Gαo. The amino terminus of the peptide forms an alpha helix that is sandwiched between switch II and the alpha3 helix of the Gαi1 helical domain. Contacts between the GoLoco domain and the switch II residues overlaps with contacts between the Gαi1 and the Gβγ subunits. The GoLoco peptide crosses the Gαi inter-domain region between the GTPase and the helical domains in a random configuration. In this region 19 conserved amino acid residues interact with both “switch 1” and GDP. The GoLoco C-terminus interacts with the A and B helices of the helical domain but at positions that do not affect the AB loop region. GoLoco binding alters the conformation of switch regions I and II this precludes the binding of Gβγ (Kimple et al., 2001 JBC 276 29275-29281).

### β-Galactosidase Enzyme Fragment Complementation

The β-galactosidase crystal structure explains α-complementation. The N-terminal donor residues (˜50) are positioned on the surface of the protein Amino acid residues 13 and 15 contribute to the activating interface and residues 29-33 pass through a “tunnel” formed by an intra-molecular domain-domain interaction. Essentially, the donor peptide threads through this tunnel and restores the interactions present in the wild-type enzyme generating enzyme activity. β-galactosidase α-complementation forms an enzyme with catalytic and substrate affinities equivalent to those of the wild-type enzyme (Olson et al., 2007 Assay & Drug Dev Tech 5, 137-144).

To generate enzyme activity a free β-galactosidase N-terminus is optimally required. Therefore this essentially controls the choice of which β-galactosidase peptide fragment is coupled to which of the protein partners. A free N-terminal peptide donor fragment can only be accommodated by coupling to the N-terminals of the RGS proteins and GoLoco domains (see FIG. 3).

Table 2 gives examples of some commercially available cDNAs.

TABLE 2
cDNAs available from Mammalian Gene Collection (MGC),
NIH, Maryland, USA

| cDNA | Species | Image clone No. | Accession No. |
|---|---|---|---|
| Gαi1 | Human | 4791696 | BC026326 |
| Gαi2 | Human | 2963802 | BC014627 |
| Gαi3 | Human | 5433334 | BC025285 |
| Gαq | Human | 6085072 | BC089041 |

| | |
|---|---|
| Gαs | Not commercially available, but various EST |
| | are described to facilitate generation |

### Technical Problem

Despite the existence of a number of non-radioactive assays, there is a continued need for new non-radioactive ligand binding assays for G protein-coupled receptors for drug screening. These assays should be highly specific and provide a clear signal which is readily detectable over background noise. Preferably, these assays should be homogeneous in nature, obviating the requirement for a washing and separation steps and making the assays suitable for compound screening purposes, particularly high throughput drug screening. More preferably, there is a need for additional live cell assays with all the benefits involved in such assays. These problems are resolved by the provision of the assay methods of the present invention, which allow monitoring of activation and deactivation of GPCRs and can be used for testing ligand binding to GPCRs.

### SUMMARY OF THE INVENTION

The present invention relates to methods, protein constructs and cells which can be used to identify molecules that modulate GPCRs and signal transduction. GPCRs transduce their signals through G-protein subunits which are in turn subject to modification by other intracellular proteins such as regulators of G-protein signalling (RGS) proteins. Enzyme fragmentation assays are described which have a luminescent end-point and which could be readily adapted for use on imaging instrumentation, such as LEADSEEKER™/VIEWLUX™ or any other luminescent-based or microtitre plate reader, using colorimetric substrates (e.g. the β-Gal substrates is commercially available from Applied Biosystems Inc).

The E. coli β-galactosidase N-terminal amino acids (e.g. residues 3-92, or smaller fragments after close inspection of crystallographic data) are used as the donor peptide and using recombinant DNA technology, nucleotides encoding these residues are attached to the RGS proteins, and/or the GoLoco domains derived from such proteins. These proteins/domains are cytosolic and all interact with Gαi.

The E. coli β-Gal acceptor peptide is engineered to remove residues 11-41. DNA sequences encoding this modified peptide is then inserted into the cDNA sequence encoding the Gαi subunit, such as the Gαi AB loop region.

In one embodiment, constructs encoding the donor peptide-RGS motif and the Gαi-acceptor peptide may be co-transfected into the appropriate cell line. Alternatively, a single construct containing an internal ribosome entry site (IRES) element could be engineered that encodes both recombinant cDNAs. Similar manipulations can be performed to generate assays for Gαq and Gαs using the cDNA encoding RGS2, RGS3 and RGS-PX1.

FIG. 3 depicts one assay format of the invention. On GPCR/heterotrimeric G-protein activation RGS4 and the GoLoco (210) domains bind to specific regions within Gαi (220) which are separate from the AB loop. This interaction will allow the β-galactosidase N-terminal amino acid donor peptide (215) to co-localise and interact with the acceptor peptide (225) residues within the Gαi AB loop. This interaction will facilitate β-galactosidase complementation and hence enzyme activity. Cell lysis and the addition of a β-galactosidase luminescent substrate (230) will produce an optical signal (240) and allow the monitoring of GPCR/G-protein activation.

According to a first aspect of the present invention, there is provided a method for testing for the binding of a ligand to a G Protein-Coupled Receptor (GPCR) in an enzyme complementation assay, the method comprising:
a) providing a fluid sample comprising a GPCR and a Gα subunit wherein the Gα subunit comprises an enzyme fragment which acts as an enzyme acceptor (EA);
b) adding a Regulator of G Protein Signalling (RGS) or a GoLoco domain of a RGS to the fluid sample wherein the RGS or the GoLoco domain comprises an enzyme fragment which acts as an enzyme donor (ED) which is capable of enzyme complementation with the EA;
c) adding a ligand to the fluid sample to allow binding of the ligand to the GPCR to promote association between the Gα subunit and the RGS or the Gα subunit and the GoLoco domain and thereby enable enzyme complementation between the EA and the ED to form an active enzyme;
d) adding a substrate of the active enzyme to the fluid sample; and
e) detecting a change in an optical signal resulting from the activity of the active enzyme on the substrate as a measure of ligand binding.

In one embodiment, the enzyme fragment is an enzyme acceptor (EA) or enzyme donor (ED) selected from the group of enzymes consisting of β-galactosidase, β-lactamase, dihydrofolate reductase, luciferase, ubiquitinase, alkaline phosphatase and tryptophan synthase.

In another embodiment, the enzyme acceptor (EA) is a fragment of β-galactosidase and the enzyme donor (ED) is a fragment of β-galactosidase.

In a further embodiment, the RGS is selected from the group consisting of RGS3, RGS4, RGS12, RGS14 and RGS-PX1.

Soundarararjan M et al., 2008, (PNAS105, 6457-6462) demonstrated that RGS2 is selective for Gαq. The authors also demonstrated that RGS3 and RGS4 are selective for Gαq and Gαi1, while RGS12 and RGS14 are selective for Gαi1. RGS-PX1 is Gαs specific. Therefore the use of RGS3, 4, 12, 14 and PX1 enables assays to be developed that facilitate monitoring the activation of a wide range of GPCRs. The selectivity exhibited by RGS proteins for specific Gα proteins is based upon differential affinities e.g. RGS4 interacts with higher affinity than RGS2 for Gαi (Heximer S. et al., 1999. J. Biol. Chem. 276, 14195-14203).

Preferably, the RGS is RGS4.

In one embodiment, the GoLoco domain is the GoLoco domain of RGS12 or the GoLoco domain of RGS14.

In another aspect, the RGS is RGS4, the enzyme acceptor (EA) is a fragment of β-galactosidase and the enzyme donor (ED) is a fragment of β-galactosidase.

In yet another embodiment, the GoLoco domain is the GoLoco domain of RGS14, the enzyme acceptor (EA) is a fragment of β-galactosidase and the enzyme donor (ED) is a fragment of β-galactosidase.

In a further embodiment, the GPCR is in the form of a membrane preparation.

In another embodiment, the method is an homogeneous assay.

In one embodiment, the optical signal is a luminescent signal or a fluorescent signal. Preferably the optical signal is a luminescent signal.

According to a second aspect of the present invention, there is provided a cell-based assay for testing for the binding of a ligand to a G Protein Coupled Receptor (GPCR) in an enzyme complementation assay, the method comprising:
a) providing a cell expressing a GPCR and a Gα subunit wherein the Gα subunit comprises an enzyme fragment which acts as an enzyme acceptor (EA);
b) the cell further expressing a Regulator of G Protein Signalling (RGS) or a GoLoco domain of a RGS wherein the RGS or the GoLoco domain comprises an enzyme fragment which acts as an enzyme donor (ED) which is capable of enzyme complementation with the EA;
c) adding a ligand to the cell to allow binding of the ligand to the GPCR to promote association between the RGS or the GoLoco domain and the Gα subunit and thereby enable enzyme complementation between the enzyme donor (ED) and the enzyme acceptor (EA) to form an active enzyme;
d) lysing the cell to provide a cellular lysate;
e) adding a substrate of the active enzyme to the cellular lysate; and
f) detecting a change in an optical signal resulting from the activity of the active enzyme on the substrate as a measure of ligand binding.

In one embodiment, the enzyme fragment is an enzyme acceptor (EA) or enzyme donor (ED) selected from the group of enzymes consisting of β-galactosidase, β-lactamase, dihydrofolate reductase, luciferase, ubiquitinase, alkaline phosphatase and trytophan synthase.

In another embodiment, the enzyme acceptor (EA) is a fragment of β-galactosidase and the enzyme donor (ED) is a fragment of β-galactosidase.

In a further embodiment, the RGS is selected from the group consisting of RGS3, RGS4, RGS12, RGS14 and RGS-PX1. As discussed above, Soundarararjan M et al., 2008, (PNAS105, 6457-6462) demonstrated that RGS2 is selective for Gαq. The authors also demonstrated that RGS3 and RGS4 are selective for Gαq and Gαi1, while RGS12 and RGS14 are selective for Gαi1. RGS-PX1 is Gαs specific. Therefore the use of RGS3, 4, 12, 14 and PX1 enables assays to be developed that facilitate monitoring the activation of a wide range of GPCRs.

Preferably the RGS is RGS4.

In one embodiment, the RGS is RGS4, the enzyme acceptor (EA) is a fragment of β-galactosidase and the enzyme donor (ED) is a fragment of β-galactosidase.

In another embodiment, the GoLoco domain is the GoLoco domain of RGS12 or the GoLoco domain of RGS14.

In yet another embodiment, the GoLoco domain is the GoLoco domain of RGS14, the enzyme acceptor (EA) is a fragment of β-galactosidase and the enzyme donor (ED) is a fragment of β-galactosidase.

In one embodiment, the optical signal is a luminescent signal or a fluorescent signal. Preferably the optical signal is a luminescent signal.

In a third aspect of the present invention, there is provided a cell expressing
a) a G Protein Coupled Receptor (GPCR);
b) a Gα subunit comprising an enzyme fragment which acts as an enzyme acceptor (EA); and
c) a Regulator of G Protein Signalling (RGS) or a GoLoco domain thereof wherein the RGS or said GoLoco domain comprises an enzyme fragment which acts as an enzyme donor (ED) which is capable of enzyme complementation with the EA.

In one embodiment, the enzyme fragment is an enzyme acceptor (EA) or an enzyme donor (ED) selected from the group of enzymes consisting of β-galactosidase, β-lactamase, dihydrofolate reductase, luciferase, ubiquitinase, alkaline phoshpatase and tryptophan synthase.

In another embodiment, the enzyme acceptor (EA) is a fragment of β-galactosidase and the enzyme donor (ED) is a fragment of β-galactosidase.

In a further embodiment, the RGS is selected from the group consisting of RGS3, RGS4, RGS12, RGS14 and RGS-PX1. As discussed above, Soundarararjan M et al., 2008, (PNAS105, 6457-6462) demonstrated that RGS2 is selective for Gαq. The authors also demonstrated that RGS3 and RGS4 are selective for Gαq and Gαi1, while RGS12 and RGS14 are selective for Gαi1. RGS-PX1 is Gαs specific. Therefore the use of RGS3, 4, 12, 14 and PX1 enables assays to be developed that facilitate monitoring the activation of a wide range of GPCRs.

In one embodiment, the RGS is RGS4, the enzyme acceptor (EA) is a fragment of β-galactosidase and the enzyme donor (ED) is a fragment of β-galactosidase.

In another embodiment, the GoLoco domain is the GoLoco domain of RGS12 or the GoLoco domain of RGS14.

In one embodiment, the GoLoco domain is the GoLoco domain of RGS14, the enzyme acceptor (EA) is a fragment of β-galactosidase and the enzyme donor (ED) is a fragment of β-galactosidase.

According to a fourth aspect of the present invention, there is provided a protein construct comprising a Gα subunit comprising an enzyme fragment which acts as an enzyme acceptor (EA) which is capable of enzyme complementation with an enzyme donor (ED).

In a fifth aspect of the present invention, there is provided a protein construct comprising a Regulator of G Protein Signalling (RGS) or a GoLoco domain thereof wherein the RGS or the GoLoco domain comprises an enzyme fragment which acts as an enzyme donor which is capable of enzyme complementation with an enzyme acceptor. Preferably, the protein construct is selected from the group wherein RGS is selected from the group consisting of RGS3, RGS4, RGS12, RGS14 and RGS-PX1. The use of RGS3, 4, 12, 14 and PX1 enables assays to be developed that facilitate monitoring the activation of a wide range of GPCRs.

In a sixth aspect of the present invention, there is provided a nucleic acid encoding a protein construct as hereinbefore described.

According to a seventh aspect of the present invention, there is provided a vector comprising a nucleic acid as hereinbefore described.

In an eighth aspect of the present invention, there is provided a kit comprising a vector as hereinbefore described and a substrate for an enzyme complementation assay. Preferably the substrate is a substrate of an enzyme selected from the group consisting of β-galactosidase, β-lactamase, dihydrofolate reductase, luciferase, ubiquitinase, alkaline phosphatase and trytophan synthase. More preferably, the substrate is a β-galactosidase substrate.

According to a ninth aspect of the present invention, there is provided a use of a cell as hereinbefore described in drug screening.

### SEQUENCE LISTING

SEQ ID NO: 1 is the amino acid sequence of an enzyme donor (ED) of β-galactosidase.
SEQ ID NO: 2 is the amino acid sequence of an enzyme acceptor (EA) of β-galactosidase.
SEQ ID NO: 3 is a nucleotide sequence encoding the β-galactosidase enzyme donor (ED) peptide of SEQ ID NO: 1.
SEQ ID NO: 4 is a nucleotide sequence encoding the β-galactosidase enzyme acceptor (EA) peptide of SEQ ID NO: 2.
SEQ ID NO: 5 is an amino acid sequence of a 47-mer β-galactosidase enzyme donor described by Olson and Eglen (Assay and Drug Development Technologies 2007, 5, 97-105).
SEQ ID NO: 6 is the amino acid sequence of β-galactosidase (NT 1-91)-RGS12 isoform 1.
SEQ ID NO: 7 is the amino acid sequence of β-galactosidase (NT 1-91)-RGS12 isoform 2.
SEQ ID NO: 8 is the amino acid sequence of β-galactosidase (NT 1-91)-RGS12 isoform 3
SEQ ID NO: 9 is the amino acid sequence of β-galactosidase (NT 1-91)-RGS12 GoLoco domain.
SEQ ID NO: 10 is the amino acid sequence of β-galactosidase (NT 1-91)-RGS14.
SEQ ID NO: 11 is the amino acid sequence of β-galactosidase (NT 1-91)-RGS14 GoLoco domain.
SEQ ID NO: 12 is the amino acid sequence of β-galactosidase (NT 1-91)-RGS 2.
SEQ ID NO: 13 is the amino acid sequence of β-galactosidase (NT 1-91)-RGS 3 isoform 1.
SEQ ID NO: 14 is the amino acid sequence of β-galactosidase (NT 1-91)-RGS 3 isoform 2.
SEQ ID NO: 15 is the amino acid sequence of β-galactosidase (NT 1-91)-RGS 3 isoform 4.
SEQ ID NO: 16 is the amino acid sequence of β-galactosidase (NT 1-91)-RGS 3 isoform 5.
SEQ ID NO: 17 is the amino acid sequence of β-galactosidase (NT 1-91)-RGS 3 isoform 6.
SEQ ID NO: 18 is the amino acid sequence of β-galactosidase (NT 1-91)-RGS 4 isoform 1.
SEQ ID NO: 19 is the amino acid sequence of β-galactosidase (NT 1-91)-RGS 4 isoform 2.
SEQ ID NO: 20 is the amino acid sequence of β-galactosidase (NT 1-91)-RGS PXI.
SEQ ID NO: 21 is the amino acid sequence of GNαi β-galactosidase (delta 1-41 CT).
SEQ ID NO: 22 is the amino acid sequence of GNαi2 β-galactosidase (delta 1-41 CT).
SEQ ID NO: 23 is the amino acid sequence of GNαi β-galactosidase (delta 1-41 CT).
SEQ ID NO: 24 is the amino acid sequence of GNαT1 β-galactosidase (delta 1-41 CT).
SEQ ID NO: 25 is the amino acid sequence of GNαT2 β-galactosidase (delta 1-41 CT).
SEQ ID NO: 26 is the amino acid sequence of GNαT β-galactosidase (delta 1-41 CT).
SEQ ID NO: 27 is the amino acid sequence of GNαq β-galactosidase (delta 1-41 CT).
SEQ ID NO: 28 is the amino acid sequence of GNα15 (GNαq)-β-galactosidase (delta 1-41 CT).
SEQ ID NO: 29 is the amino acid sequence of GNαQ (GNα11)-β-galactosidase (delta 1-41 CT).
SEQ ID NO: 30 is the amino acid sequence of GNαS (isoform A)-β-galactosidase (delta 1-41 CT).
SEQ ID NO: 31 is the amino acid sequence of GNαS (isoform B)-β-galactosidase (delta 1-41 CT).
SEQ ID NO: 32 is the amino acid sequence of GNαS (isoform F)-β-galactosidase (delta 1-41 CT).
SEQ ID NO: 33 is the amino acid sequence of GNαS (isoform G)-β-galactosidase (delta 1-41 CT).
SEQ ID NO: 34 is the amino acid sequence of GNαS Olfactory (isoform 1)-β-galactosidase (delta 1-41 CT).
SEQ ID NO: 35 is the amino acid sequence of GNαS Olfactory (isoform 2)-β-galactosidase (delta 1-41 CT).
SEQ ID NO: 36 is the nucleotide sequence of vector pCORON 1000.
SEQ ID NO: 37 is the nucleotide sequence of vector pCORON 1000 β-galactosidase (NT 1-91)-RGS12 isoform 1.
SEQ ID NO: 38 is the nucleotide sequence of vector pCORNON 1000 GNαi1-β-galactosidase (delta 1-41 CT).
SEQ ID NO: 39 is the nucleotide sequence of vector pIRES.
SEQ ID NO: 40 is the nucleotide sequence of pIRES β-galactosidase (NT 1-91) RGS12 isoform 1-GNαi1 β-galactosidase (delta 1-41 CT).
SEQ ID NO: 41 is the amino acid sequence of a linker peptide which is included in SEQ ID NOs: 6-35. The function of this peptide is to act as a flexible link that connects naturally independent peptides moieties thereby generating a single recombinant chimeric fusion protein. The skilled person will appreciate that other suitable linker peptides could be used to carry out this function.
SEQ ID NO: 42 is the nucleotide sequence encoding the linker peptide of SEQ ID NO: 41.

“β-galactosidase (NT 1-91)”, with respect to the above sequence listings and in particular to SEQ ID NOs: 6-20, 37 and 40, refers to a short polypeptide sequence consisting of the amino acid residues 1-91 derived from the N-terminal region of the E. coli β-galactosidase.

“β-galactosidase (delta 1-41 CT)”, with respect to the above sequence listings and in particular to SEQ ID NOs: 21-35, 38 and 40, refers to a polypeptide sequence derived from the E. coli β-galactosidase that lacks residues 1-41 from the N-terminal region compared to the intact polypeptide.

### BRIEF DESCRIPTION OF THE DRAWINGS

FIG. 1 is a schematic representation of a RGS4-Gαi1 complex in accordance with the present invention.

FIG. 2 is a schematic representation of a RGS14 GoLoco-Gαi1 complex in accordance with the present invention.

FIG. 3 is a schematic representation illustrating an assay in accordance with the present invention.

FIG. 4 depicts the primary structure of β-galactosidase (NT 1-91)-RGS12 isoform 1, isoform 2 and isoform 3 (FIGS. 4a, b & c, respectively).

FIG. 5 illustrates the primary structure of β-galactosidase (NT 1-91)-RGS12 GoLoco domain.

FIG. 6 depicts the primary structure of β-galactosidase (NT 1-91)-RGS14.

FIG. 7 illustrates the primary structure of β-galactosidase (NT 1-91)-RGS14 GoLoco domain.

FIG. 8 shows the primary structure of β-galactosidase (NT 1-91)-RGS 2.

FIG. 9 shows the primary structure of β-galactosidase (NT 1-91)-RGS 3 isoforms (isoforms 1, 2, 4, 5 and 6; FIGS. 9a, b, c, d and e, respectively).

FIG. 10 depicts the primary structure of β-galactosidase (NT 1-91)-RGS 4 isoforms (isoforms 1 and 2; FIGS. 10a and b, respectively).

FIG. 11 illustrates the primary structure of (β-galactosidase (NT 1-91)-RGS-PXI.

FIG. 12 shows the primary structure of GNAi1 β-galactosidase (delta 1-41 CT).

FIG. 13 depicts the primary structure of GNAi2 β-galactosidase (delta 1-41 CT).

FIG. 14 illustrates the primary structure of GNAi3 β-galactosidase (delta 1-41 CT).

FIG. 15 shows the primary structure of GNAT1 β-galactosidase (delta 1-41 CT).

FIG. 16 depicts the primary structure of GNAT2 β-galactosidase (delta 1-41 CT).

FIG. 17 illustrates the primary structure of GNATS β-galactosidase (delta 1-41 CT).

FIG. 18 shows the primary structure of GNAq β-galactosidase (delta 1-41 CT).

FIG. 19 depicts the primary structure of GNA15 (GNAq)-β-galactosidase (delta 1-41 CT).

FIG. 20 illustrates the primary structure of GNAQ (GNA11)-β-galactosidase (delta 1-41 CT).

FIG. 21a shows the primary structure of GNAS (isoform A)-β-galactosidase (delta 1-41 CT).

FIG. 21b depicts the primary structure of GNAS (isoform B)-β-galactosidase (delta 1-41 CT).

FIG. 21c illustrates the primary structure of GNAS (isoform F)-β-galactosidase (delta 1-41 CT).

FIG. 21d shows the primary structure of GNAS (isoform G)-β-galactosidase (delta 1-41 CT).

FIG. 22a depicts the primary structure of GNAS Olfactory (isoform 1)-(3-galactosidase (delta 1-41 CT).

FIG. 22b illustrates the primary structure of GNAS Olfactory (isoform 2)-(3-galactosidase (delta 1-41 CT).

FIG. 23 is a vector diagram of pCORON1000.

FIG. 24 is a vector diagram of pCORON1000 β-galactosidase (NT 1-91)-RGS12 isoform 1.

FIG. 25 is a vector diagram of pCORON1000 pCORON1000 GNAi1-β-galactosidase (delta 1-41 CT).

FIG. 26 is a vector diagram of pIRES.

FIG. 27 is a vector diagram of pIRES β-galactosidase (NT 1-91) RGS12 isoform 1-GNAi1 β-galactosidase (delta 1-41 CT).

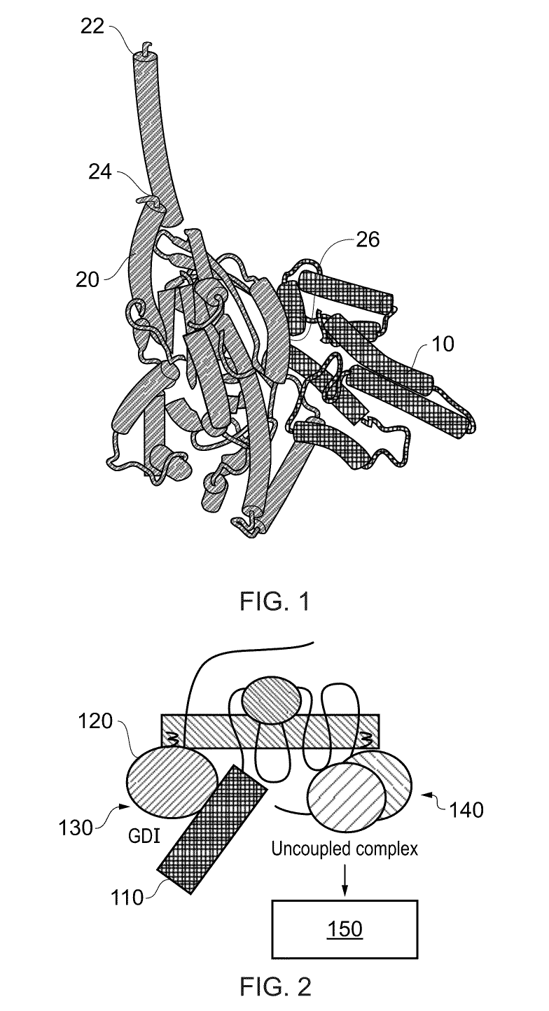

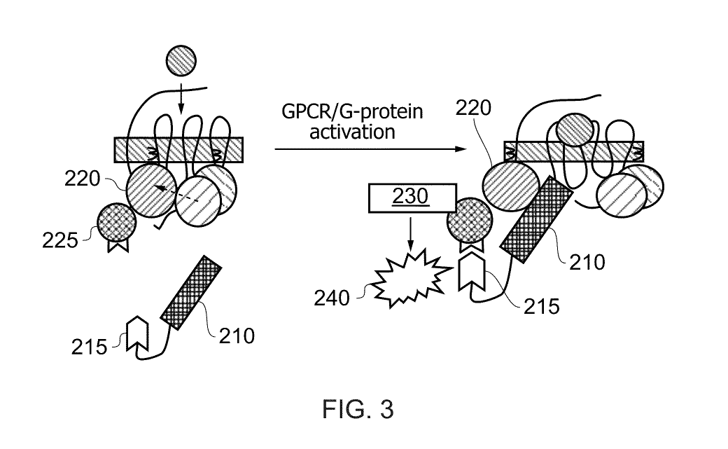

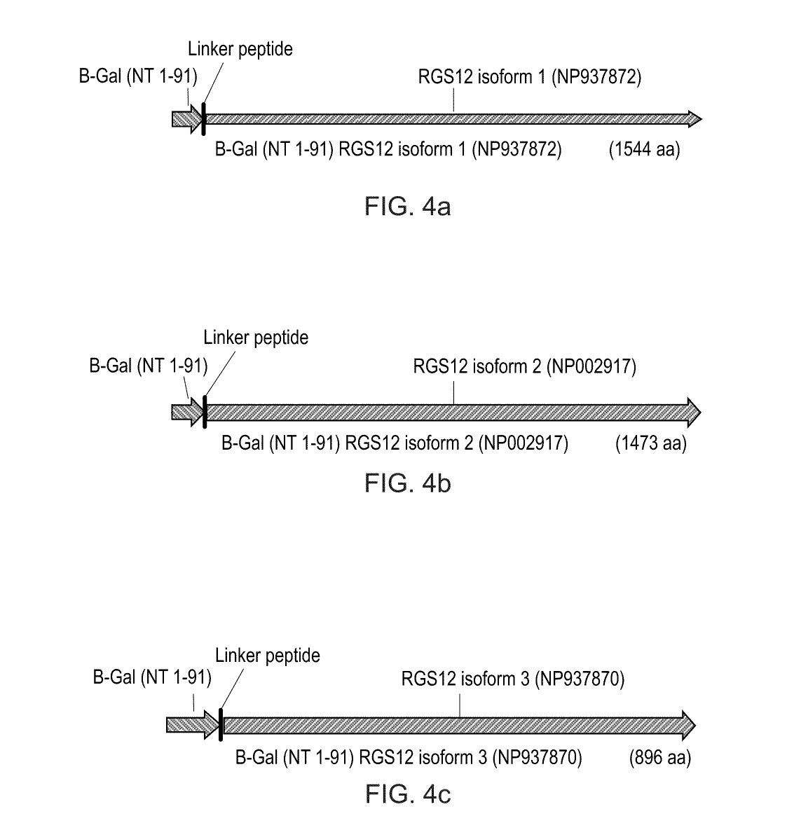

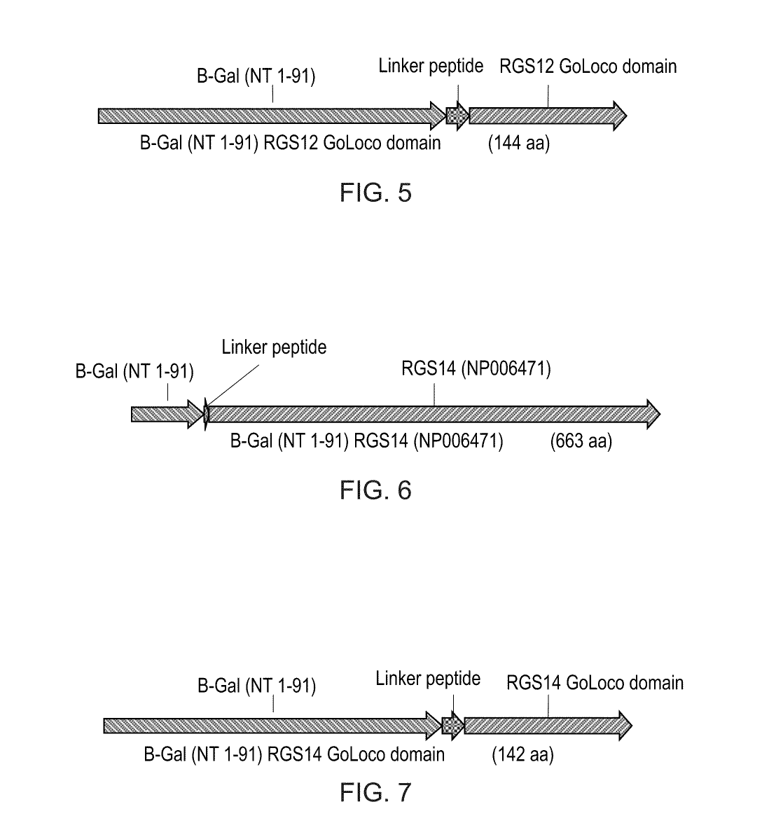

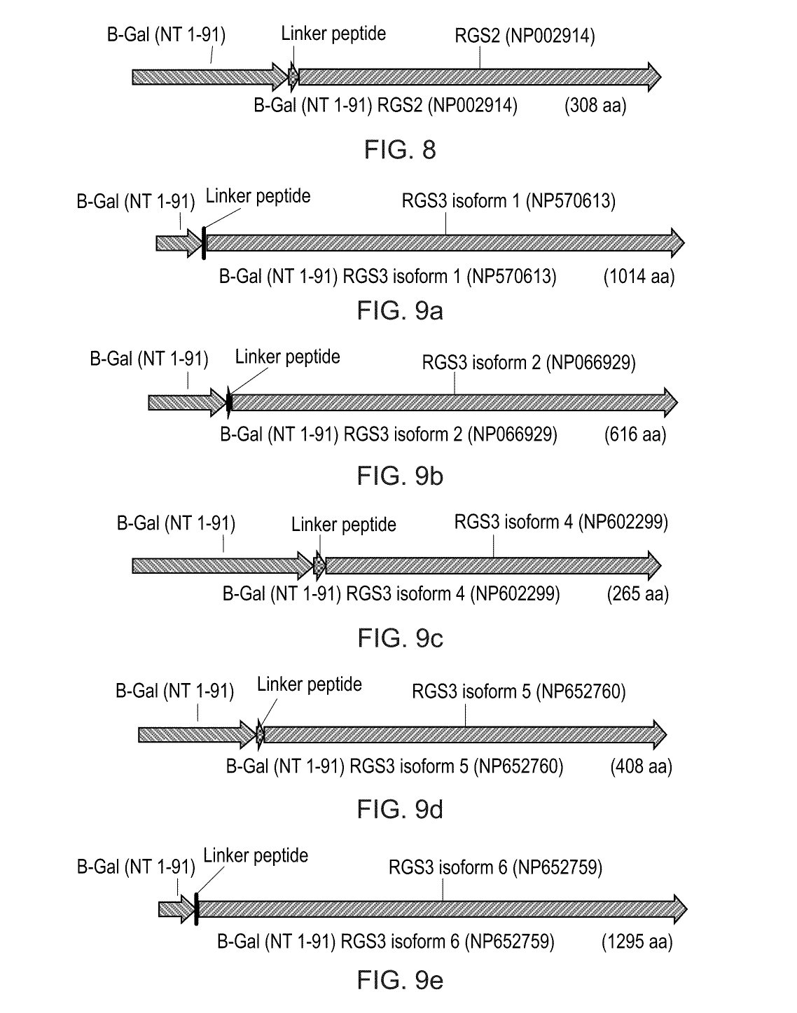

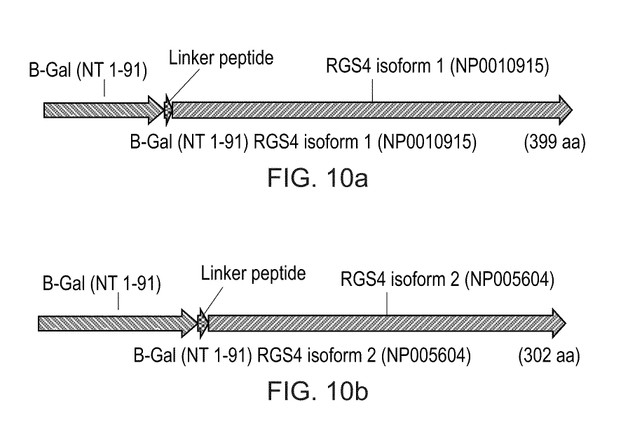

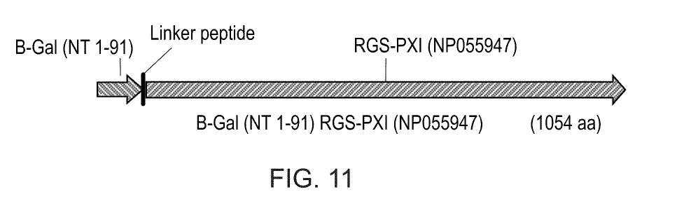

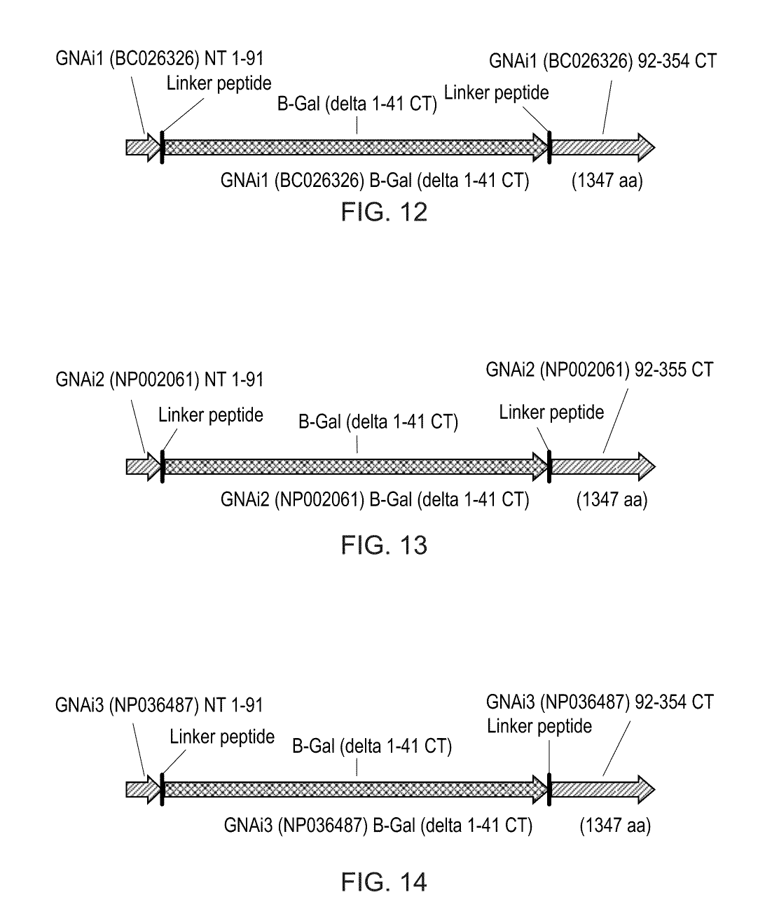

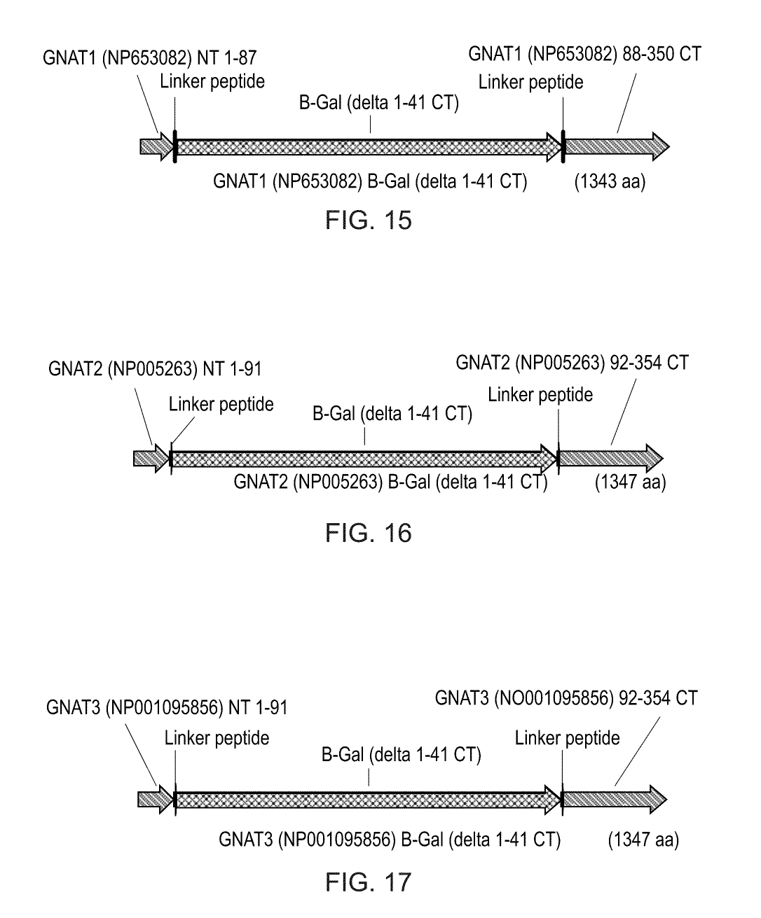

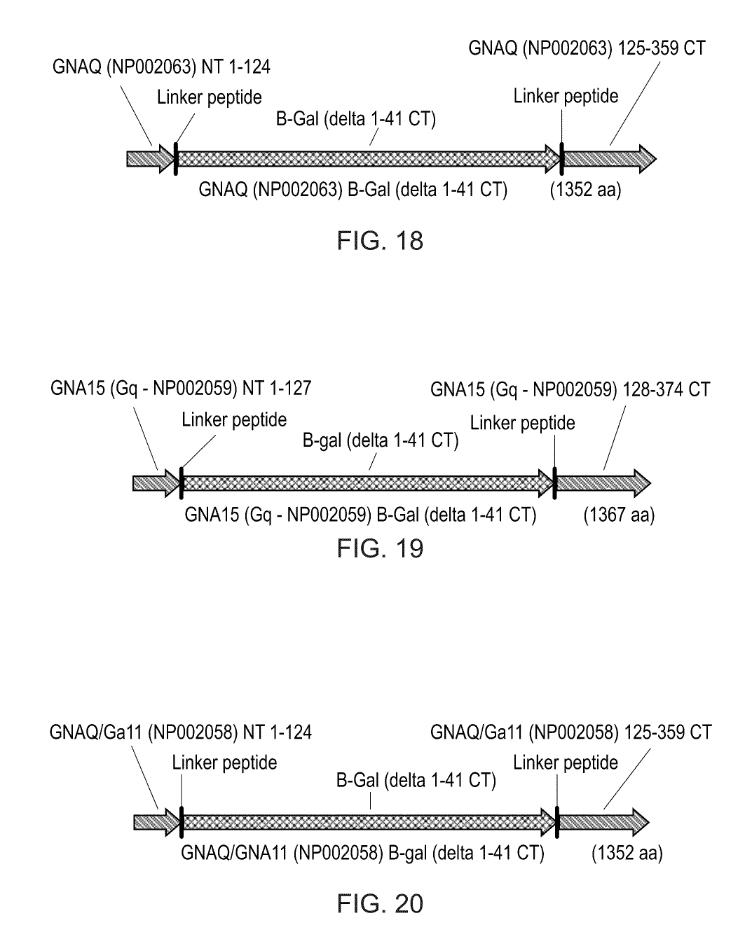

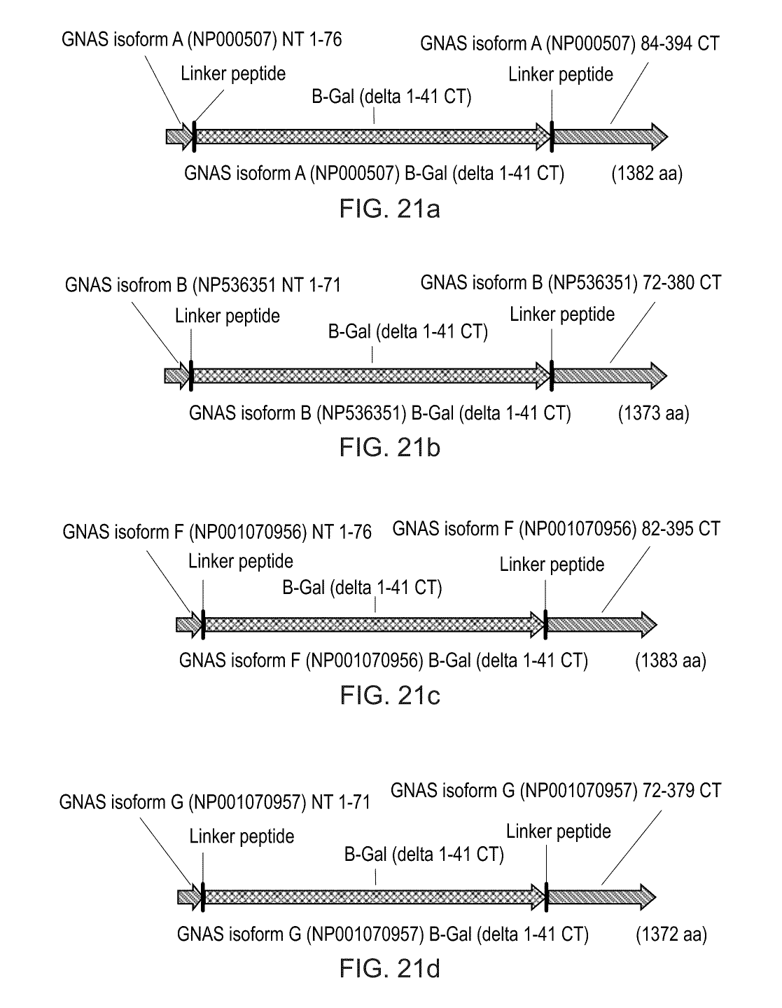

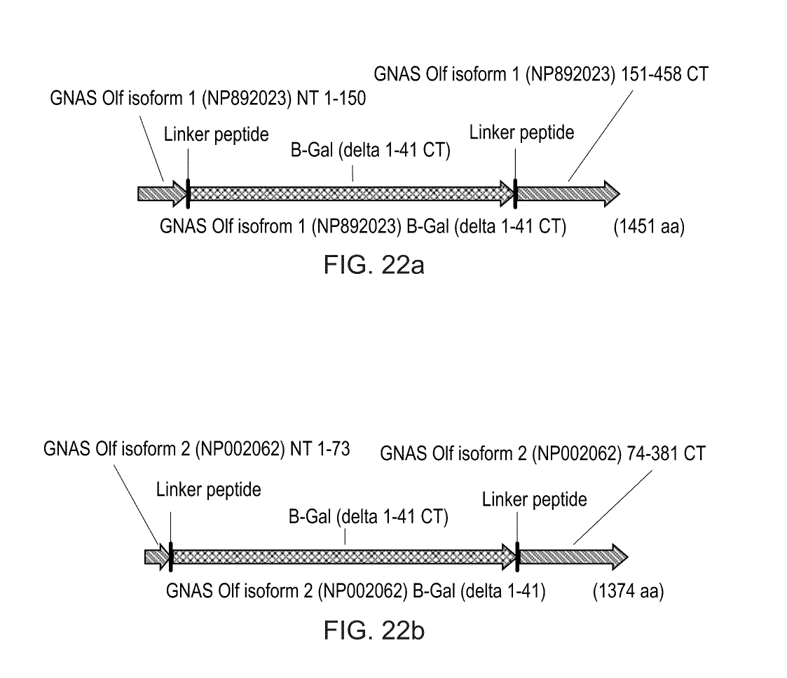

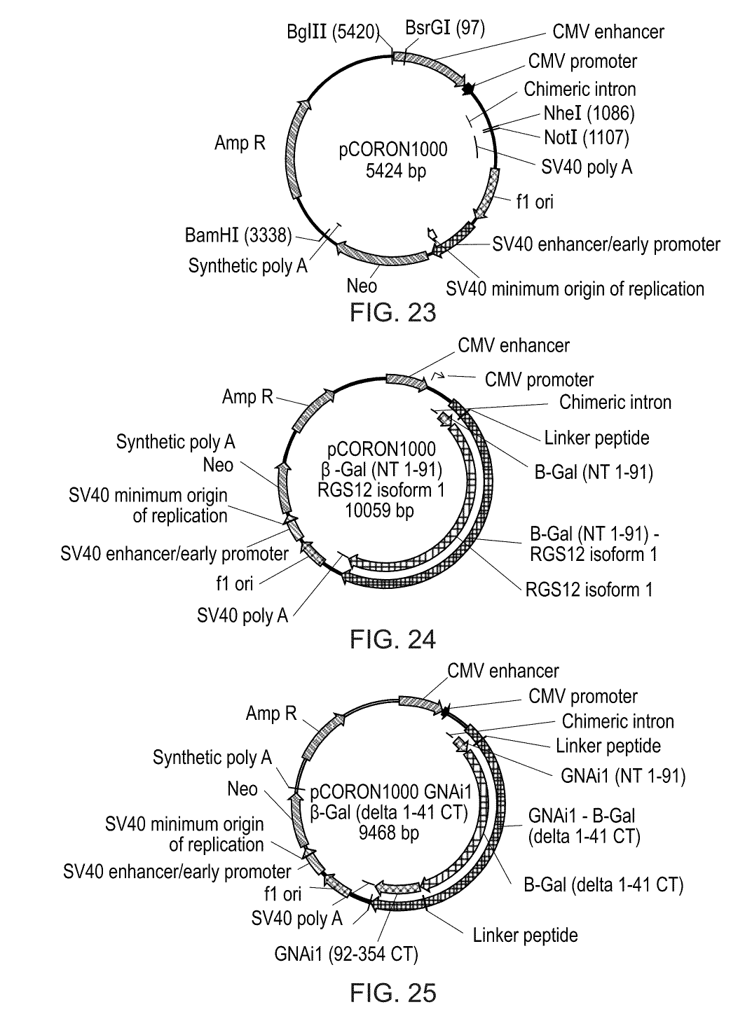

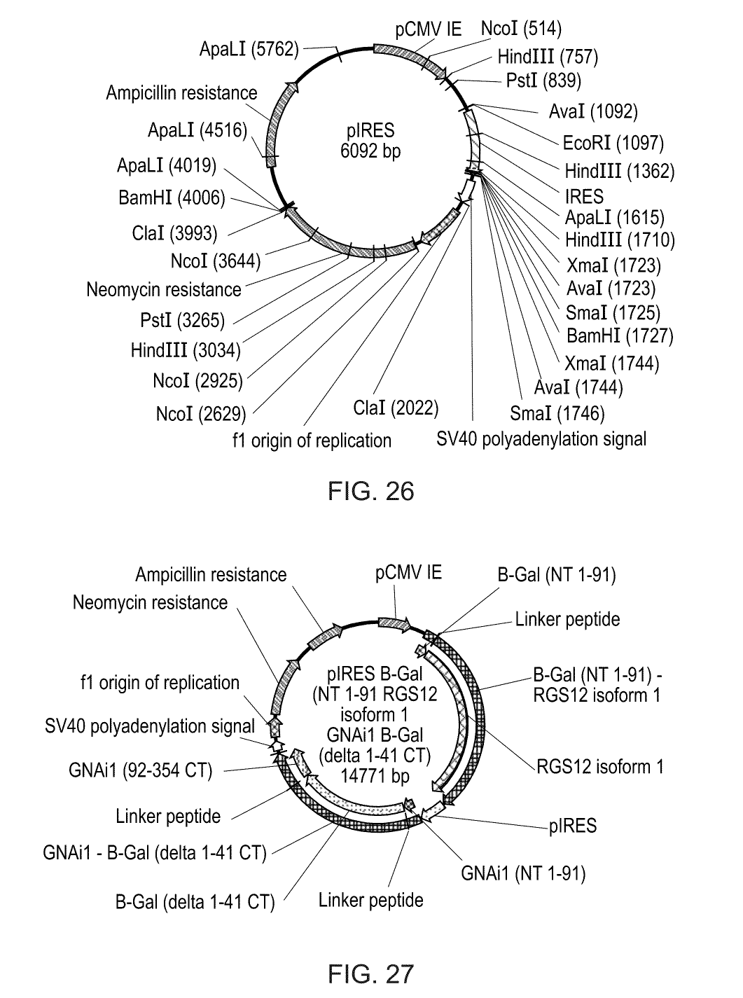

### DETAILED DESCRIPTION OF THE INVENTION

### Examples

The following examples serve to illustrate embodiments of the present invention. These examples are intended to demonstrate techniques which the present inventors have found to work well in practising the present invention. Hence these examples are detailed so as to provide those of ordinary skill in the art with a complete disclosure and description of the ways in which the methods of this invention may be performed. The following Examples are intended to be exemplary only and changes, modification and alterations can be employed to the conditions described herein, without departing from the scope of the invention.

### 1. Preparation of Genetic Constructs and Transfections of Cells.

### 1.1 Preparation of Gα Enzyme Acceptor Fragments

The method involves creation of a polypeptide chimera comprising a Gα subunit which comprises an enzyme fragment which acts as a β-galactosidase enzyme acceptor (EA) which is capable of enzyme complementation with a β-galactosidase enzyme donor (ED) fragment. The enzyme acceptor component lacks the coding for key amino acids at chosen sites of the lacZ gene, and the expressed β-galactosidase would normally exist as an enzymatically inactive dimer.

One of the more widely studied examples of a β-galactosidase EA peptide is the X90-acceptor peptide that has a deletion in the last 10 amino acids (1013-1023).

The X90 EA peptide exists as a monomer and can be complemented by a corresponding ED fragment of β-galactosidase, such as CNBr24, a cyanogen bromide digestion product of β-galactosidase consisting of amino acids 990-1023, to reform enzymatically active tetramer (Welphy et al., 1980, Biochem. Biophys. Res. Common, 93, 223).

An enzyme acceptor (EA) fragment is inserted into a Gα subunit. In one embodiment, the Gα subunit is the Gαi subunit. In other embodiments, the Gα subunit is the Gαq subunit or the Gas subunit. The amino acid sequences of suitable Gα-β galactosidase enzyme acceptor (EA) constructs which may be used in the present invention are shown in SEQ ID NOs: 21 to 35. The cDNA (full length) of many of the Gα subunit sequences are available from commercial sources (e.g. Mammalian Gene Collection (MGC), NIH, Maryland, USA, Table 2).

A vector is constructed (e.g. pCI-neo vector from Promega, Cat no. E1841) using techniques well known in the art coding for the chimera Gα/enzyme acceptor (EA). The pCI-neo Mammalian Expression Vector carries the human cytomegalovirus (CMV) immediate-early enhancer/promoter region to promote constitutive expression of cloned DNA inserts in mammalian cells. Other suitable vectors (e.g. SEQ ID NO: 38), such as those based on the pCORON vector (e.g. SEQ ID NO: 36), can also be used. This vector (i.e. as shown in SEQ ID NO: 38) also contains the neomycin phosphotransferase gene, a selectable marker for mammalian cells. The pCI-neo Vector can be used for transient expression or for stable expression by selecting transfected cells with the antibiotic G-418.

Transfection of target cells (e.g. mammalian cells) using a transfection agent such as Fugene6, with the above-described vector is carried out in accordance with Manufacturer's instructions and following the principles outlined by Sambrook and Russell (Molecular Cloning, A Laboratory Manual, 3^{rd} Edition, Volume 3, Chapter 16, Section 16.1-16.54). For example, Fugene6 and jetPEI, Roche and Polyplus Transfections respectively. In addition transient viral transduction can also be performed using reagents such as adenoviral vectors (Ng P and Graham F L. Methods Mol. Med. 2002; 69, 389-414).

The resulting transfected cells are maintained in culture or frozen for later use according to standard practices. These cells express the desired Gα-EA chimera protein, as described above.

### 1.2 Preparation of RGS and GoLoco Domain β-Galactosidase Enzyme Donor Fragments

RGS and GoLoco domain β-galactosidase enzyme donor fragments are prepared in a similar manner to that described for the Gα enzyme acceptor fragments above using standard molecular biological techniques according to Sambrook and Russell (Molecular Cloning, A Laboratory Manual).

In one embodiment of the present invention, the β-galactosidase enzyme donor fragment has the amino acid sequence shown in SEQ ID NO: 1. In another embodiment, the β-galactosidase enzyme donor fragment has the amino acid sequence shown in SEQ ID NO: 5:

### Cys Ser Leu Ala Val Val Leu Gln Arg Arg Asp Trp Glu Asn Pro Gly Val Thr Gln Leu Asn Arg Leu Ala Ala H is Pro Pro Phe Ala Ser Trp Arg Asn Ser Glu Glu Ala Arg Thr Asp Cys Pro Ser Gln Gln Leu.

The 47-mer β-galactosidase enzyme donor being described by Olson and Eglen (Assay and Drug Development Technologies 2007, 5, 97-105).

cDNA (full length) sequences of RGS (e.g. RGS2, RGS3, RGS4 and RGS-PX1) and GoLoco domains of RGS (e.g. RGS12 and RGS14) proteins are available from commercial sources (e.g. Mammalian Gene Collection (MGC), NIH, Maryland, USA, Table 2). Suitable amino acid constructs, for use in the present invention, include SEQ ID NOs: 6 to 20.

A vector is constructed (e.g. pCI-neo vector from Promega, Cat no. E1841) using techniques well known in the art coding for the chimera RGS/β-galactosidase enzyme donor fragment or GoLoco/β-galactosidase enzyme donor fragment. The pCI-neo Mammalian Expression Vector carries the human cytomegalovirus (CMV) immediate-early enhancer/promoter region to promote constitutive expression of cloned DNA inserts in mammalian cells. Other suitable vectors (e.g. SEQ ID NO: 37), such as those based on the pCORON vector (e.g. SEQ ID NO: 36), can also be used. This vector (i.e. as shown in SEQ ID NO: 37) also contains the neomycin phosphotransferase gene, a selectable marker for mammalian cells. The pCI-neo Vector can be used for transient expression or for stable expression by selecting transfected cells with the antibiotic G-418.

Transfection of target cells (e.g. mammalian cells) using a transfection agent such as Fugene6, with the above-described vector is carried out in accordance with Manufacturer's instructions and following the principles outlined by Sambrook and Russell (Molecular Cloning, A Laboratory Manual, 3^{rd} Edition, Volume 3, Chapter 16, Section 16.1-16.54). For example Fugene6 and jetPEI, Roche and Polyplus Transfections respectively. In addition transient viral transduction can also be performed using reagents such as adenoviral vectors (Ng P and Graham F L. Methods Mol. Med. 2002; 69, 389-414).

### 1.3 Preparation of Cells Expressing Dual Constructs

Cells expressing both Gα β-galactosidase enzyme acceptor fragments and RGS β-galactosidase or GoLoco domain β-galactosidase enzyme donor fragments are prepared by co-transfecting cells with the vectors described in 1.1 and 1.2 above (or the pIRES vector shown in SEQ ID NO: 40).

The expression vectors described herein allows for the generation of stable cell lines by techniques well known in the art.

### 2. GPCR Assays

Methods of carrying out GPCR assays are well documented in the literature and are well known in the art.

By way of illustration only, and without limitation to the specific assays disclosed, the following techniques are described to demonstrate different assays for utilising the methods of the invention to test for ligand binding to a GPCR.

### 2.1 Assay Methods

Intact cells expressing a GPCR, a RGS protein which comprises a β-galactosidase enzyme donor (ED) fragment and a Gα subunit comprising a β-galactosidase enzyme acceptor (EA) are allowed to come into contact in a tube (microwell) in the presence of a suitable buffer. In the presence of a suitable GPCR ligand (e.g. isoproterenol, noradrenaline, salmeterol, denopamine etc.) the GPCR becomes activated, leading to a close proximity of the Gα-EA and the RGS-ED fragments which will lead to β-galactosidase enzyme complementation. Upon lysis of the cells, with a suitable lysing agent (e.g. TRITON® X-100 or TWEEN™ 20) and addition of a suitable β-galactosidase substrate such as the pro-luminescent 1,2-dioxetane substrate (alternative substrates include, for example, 5-acetylaminofluorescein di-b-D-galactopyranoside (X-gal) from Invitrogen; 5-Iodo-3-indolyl-beta-D-galactopyranoside from Sigma; or 5-acetylaminofluorescein di-b-D-galactopyranoside from Invitrogen), an optical signal is generated which can be detected by, for example, a photomultiplier device or a CCD camera. The optical signal can be, for example, a luminescent or a fluorescent signal.

In this system, a signal increase arises from a higher degree of β-galactosidase complementation which is directly proportional to the potency of activation of the ligand.

It will be understood that this method can be adapted to use recombinant proteins in an acellular approach using a cell-free system utilising cell membranes.

### 2.2 Screening Assay Method for GPCR Activation

Cells which express the appropriate combination of constructs described in section 1.3 above are transferred into a 96 (20,000 pre well) or 384 (5,000 cells per well) well culture plate and incubated overnight at 37° C. in a 5% atmosphere of CO₂. An aliquot (e.g. 5 μl) of a suitable test compound or ligand (e.g. isoproterenol, noradrenaline, salmeterol, denopamine etc.) dissolved or suspended in a non-toxic solvent is added to each well and the plate incubated for 1 hour at 37° C. in a 5% atmosphere of CO₂ to allow enzyme complementation to occur. A lysis reagent (such as an appropriate detergent, e.g. TRITON® X-100 or TWEEN™ 20) is added to each well and the plate incubated for 5 minutes. An appropriate luminescent substrate of β-galactosidase (e.g. 5-acetylaminofluorescein di-b-D-galactopyranoside (X-gal) from Invitrogen; 5-Iodo-3-indolyl-beta-D-galactopyranoside from Sigma; or 5-acetylaminofluorescein di-b-D-galactopyranoside from Invitrogen) is added to each well and the plate incubated for 1 to 18 hour (s) at 37° C. in a 5% CO₂ atmosphere. A change in the optical signal (e.g. fluorescence or luminescence) is read using a plate reader, imager (e.g. LEADSEEKER™ GE Healthcare) or CCD camera.

While preferred illustrative embodiments of the present invention are described, one skilled in the art will appreciate that the present invention can be practised by other than the described embodiments, which are presented for purposes of illustration only and not by way of limitation. The present invention is limited only by the claims that follow.

1. A method for testing for the binding of a ligand to a G Protein-Coupled Receptor (GPCR) in an enzyme complementation assay, said method comprising:
a) providing a fluid sample comprising a GPCR and a Gα subunit wherein said Gα subunit comprises an enzyme fragment which acts as an enzyme acceptor (EA);
b) adding a Regulator of G Protein Signalling (RGS) selected from the group consisting of RGS3, RGS4, RGS12, RGS14 and RGS-PX11 or a GoLoco domain of a RGS to said fluid sample wherein said RGS or said GoLoco domain comprises an enzyme fragment which acts as an enzyme donor (ED) which is capable of enzyme complementation with said EA;
c) adding a ligand to the fluid sample to allow binding of said ligand to the GPCR to promote association between the Gα subunit and the RGS or the Gα subunit and the GoLoco domain and thereby enable enzyme complementation between the EA and said ED to form an active enzyme;
d) adding a substrate of said active enzyme to the fluid sample; and
e) detecting a change in an optical signal resulting from the activity of the active enzyme on said substrate as a measure of ligand binding.

2. The method of claim 1, wherein said enzyme fragment is an enzyme acceptor (EA) or enzyme donor (ED) selected from the group of enzymes consisting of β-galactosidase, β-lactamase, dihydrofolate reductase, luciferase, ubiquitinase, alkaline phosphatase and tryptophan synthase.

3. The method of claim 1, wherein the enzyme acceptor (EA) is a fragment of β-galactosidase and the enzyme donor (ED) is a fragment of β-galactosidase.

4-5. (canceled)

6. The method of claim 1, wherein the GoLoco domain is the GoLoco domain of RGS12 or the GoLoco domain of RGS14.

7. (canceled)

8. The method of claim 6, wherein the GoLoco domain is the GoLoco domain of RGS14, the enzyme acceptor (EA) is a fragment of β-galactosidase and the enzyme donor (ED) is a fragment of β-galactosidase.

9. (canceled)

10. The method of claim 1, wherein said method is an homogeneous assay.

11. (canceled)

12. A cell-based assay for testing for the binding of a ligand to a G Protein Coupled Receptor (GPCR) in an enzyme complementation assay, said method comprising:
a) providing a cell expressing a GPCR and a Gα subunit wherein said Gα subunit comprises an enzyme fragment which acts as an enzyme acceptor (EA);
b) said cell further expressing a Regulator of G Protein Signalling (RGS) selected from the group consisting of RGS3, RGS4, RGS12, RGS14 and RGS-PX1 or a GoLoco domain of a RGS wherein said RGS or said GoLoco domain comprises an enzyme fragment which acts as an enzyme donor (ED) which is capable of enzyme complementation with said enzyme acceptor (EA);
c) adding a ligand to the cell to allow binding of said ligand to the GPCR to promote association between said RGS or said GoLoco domain and the Gα subunit and thereby enable enzyme complementation between said enzyme donor (ED) and the enzyme acceptor (EA) to form an active enzyme;
d) lysing the cell to provide a cellular lysate;
e) adding a substrate of said active enzyme to said cellular lysate; and
f) detecting a change in an optical signal resulting from the activity of the active enzyme on said substrate as a measure of ligand binding.

13. The method of claim 12, wherein the enzyme fragment is an enzyme acceptor (EA) or enzyme donor (ED) selected from the group of enzymes consisting of β-galactosidase, β-lactamase, dihydrofolate reductase, luciferase, ubiquitinase, alkaline phosphatase and trytophan synthase.

14. The method of claim 13, wherein the enzyme acceptor (EA) is a fragment of β-galactosidase and the enzyme donor (ED) is a fragment of β-galactosidase.

15-17. (canceled)

18. The method of claim 12, wherein the GoLoco domain is the GoLoco domain of RGS12 or the GoLoco domain of RGS14.

19. The method of claim 18, wherein the GoLoco domain is the GoLoco domain of RGS14, the enzyme acceptor (EA) is a fragment of β-galactosidase and the enzyme donor (ED) is a fragment of β-galactosidase.

20. (canceled)

21. A cell expressing
a) a G Protein Coupled Receptor (GPCR);
b) a Gα subunit comprising an enzyme fragment which acts as an enzyme acceptor (EA); and
c) a Regulator of G Protein Signalling (RGS) selected from the group consisting of RGS3, RGS4, RGS12, RGS14 and RGS-PX1 or a GoLoco domain thereof wherein said RGS or said GoLoco domain comprises an enzyme fragment which acts as an enzyme donor (ED) which is capable of enzyme complementation with said enzyme acceptor (EA).

22. The cell of claim 21, wherein said enzyme fragment is an enzyme acceptor (EA) or an enzyme donor (ED) selected from the group of enzymes consisting of β-galactosidase, β-lactamase, dihydrofolate reductase, luciferase, ubiquitinase, alkaline phoshpatase and tryptophan synthase.

23. The cell of claim 21, wherein the enzyme acceptor (EA) is a fragment of β-galactosidase and the enzyme donor (ED) is a fragment of β-galactosidase.

24-25. (canceled)

26. The cell of claim 21, wherein the GoLoco domain is the GoLoco domain of RGS12 or the GoLoco domain of RGS14.

27. The cell of claim 26, wherein the GoLoco domain is the GoLoco domain of RGS14, the enzyme acceptor (EA) is a fragment of β-galactosidase and the enzyme donor (ED) is a fragment of β-galactosidase.

28. (canceled)

29. A protein construct comprising a Regulator of G Protein Signalling (RGS) selected from the group consisting of RGS3, RGS4, RGS12, RGS14 and RGS-PX1 or a GoLoco domain thereof wherein said RGS or said GoLoco domain comprises an enzyme fragment which acts as an enzyme donor (ED) which is capable of enzyme complementation with an enzyme acceptor (EA).

30. (canceled)

31. A nucleic acid encoding the protein construct of claim 29.

32. A vector comprising the nucleic acid of claim 31.

33. A kit comprising the vector of claim 32 and a substrate for an enzyme complementation assay.

34. (canceled)


SEQUENCE LISTING (42 sequences)

SEQ ID NO: 1 - protein, 91 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQM

SEQ ID NO: 2 - protein, 981 aa, Artificial Sequence
  Synthetic peptide
RTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 3 - DNA, 273 bp, Artificial Sequence
  Synthetic oligonulceotide
atgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatg

SEQ ID NO: 4 - DNA, 2946 bp, Artificial Sequence
  Synthetic Oligonucleotide
cgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 5 - protein, 47 aa, Artificial sequence
  Synthetic peptide
CSLAVVLQRRDWENPGVTQLNRLAAHPPFASWRNSEEARTDCPSQQL

SEQ ID NO: 6 - protein, 1544 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMFRAGEASKRPLPGPSPPRVRSVEVARGRAGYGFTLSGQAPCVLSCVMRGSPADFVGLRAGDQILAVNEINVKKASHEDVVKLIGKCSGVLHMVIAEGVGRFESCSSDEEGGLYEGKGWLKPKLDSKALGINRAERVVEEMQSGGIFNMIFENPSLCASNSEPLKLKQRSLSESAATRFDVGHESINNPNPNMLSKEEISKVIHDDSVFSIGLESHDDFALDASILNVAMIVGYLGSIELPSTSSNLESDSLQAIRGCMRRLRAEQKIHSLVTMKIMHDCVQLSTDKAGVVAEYPAEKLAFSAVCPDDRRFFGLVTMQTNDDGSLAQEEEGALRTSCHVFMVDPDLFNHKIHQGIARRFGFECTADPDTNGCLEFPASSLPVLQFISVLYRDMGELIEGMRARAFLDGDADAHQNNSTSSNSDSGIGNFHQEEKSNRVLVVDLGGSSSRHGPGGSAWDGVGGRGAQPWGAPWTGPFCPDPEGSPPFEAAHQTDRFWDLNKHLGPASPVEVPPASLRSSVPPSKRGTVGAGCGFNQRWLPVHVLREWQCGHTSDQDSYTDSTDGWSSINCGTLPPPMSKIPADRYRVEGSFAQPPLNAPKREWSRKAFGMQSIFGPHRNVRKTKEDKKGSKFGRGTGLTQPSQRTSARRSFGRSKRFSITRSLDDLESATVSDGELTGADLKDCVSNNSLSSNASLPSVQSCRRLRERRVASWAVSFERLLQDPVGVRYFSDFLRKEFSEENILFWQACEYFNHVPAHDKKELSYRAREIFSKFLCSKATTPVNIDSQAQLADDVLRAPHPDMFKEQQLQIFNLMKFDSYTRFLKSPLYQECILAEVEGRALPDSQQVPSSPASKHSLGSDHSSVSTPKKLSGKSKSGRSLNEELGDEDSEKKRKGAFFSWSRTRSTGRSQKKREHGDHADDALHANGGLCRRESQGSVSSAGSLDLSEACRTLAPEKDKATKHCCIHLPDGTSCVVAVKAGFSIKDILSGLCERHGINGAAADLFLVGGDKPLVLHQDSSILESRDLRLEKRTLFRLDLVPINRSVGLKAKPTKPVTEVLRPVVARYGLDLSGLLVRLSGEKEPLDLGAPISSLDGQRVVLEEKDPSRGKASADKQKGVPVKQNTAVNSSSRNHSATGEERTLGKSNSIKIKGENGKNARDPRLSKREESIAKIGKKKYQKINLDEAEEFFELISKAQSNRADDQRGLLRKEDLVLPEFLRLPPGSTELTLPTPAAVAKGFSKRSATGNGRESASQPGEQWEPVQESSDSPSTSPGSASSPPGPPGTTPPGQKSPSGPFCTPQSPVSLAQEGTAQIWKRQSQEVEAGGIQTVEDEHVAELTLMGEGDISSPNSTLLPPPSTPQEVPGPSRPGSGTHGSRDLPVNRIIDVDLVTGSAPGRDGGIAGAQAGPGRSQASGGPPTSDLPGLGPVPGEPAKPKTSAHHATFV

SEQ ID NO: 7 - protein, 1473 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMFRAGEASKRPLPGPSPPRVRSVEVARGRAGYGFTLSGQAPCVLSCVMRGSPADFVGLRAGDQILAVNEINVKKASHEDVVKLIGKCSGVLHMVIAEGVGRFESCSSDEEGGLYEGKGWLKPKLDSKALGINRAERVVEEMQSGGIFNMIFENPSLCASNSEPLKLKQRSLSESAATRFDVGHESINNPNPNMLSKEEISKVIHDDSVFSIGLESHDDFALDASILNVAMIVGYLGSIELPSTSSNLESDSLQAIRGCMRRLRAEQKIHSLVTMKIMHDCVQLSTDKAGVVAEYPAEKLAFSAVCPDDRRFFGLVTMQTNDDGSLAQEEEGALRTSCHVFMVDPDLFNHKIHQGIARRFGFECTADPDTNGCLEFPASSLPVLQFISVLYRDMGELIEGMRARAFLDGDADAHQNNSTSSNSDSGIGNFHQEEKSNRVLVVDLGGSSSRHGPGGSAWDGVGGRGAQPWGAPWTGPFCPDPEGSPPFEAAHQTDRFWDLNKHLGPASPVEVPPASLRSSVPPSKRGTVGAGCGFNQRWLPVHVLREWQCGHTSDQDSYTDSTDGWSSINCGTLPPPMSKIPADRYRVEGSFAQPPLNAPKREWSRKAFGMQSIFGPHRNVRKTKEDKKGSKFGRGTGLTQPSQRTSARRSFGRSKRFSITRSLDDLESATVSDGELTGADLKDCVSNNSLSSNASLPSVQSCRRLRERRVASWAVSFERLLQDPVGVRYFSDFLRKEFSEENILFWQACEYFNHVPAHDKKELSYRAREIFSKFLCSKATTPVNIDSQAQLADDVLRAPHPDMFKEQQLQIFNLMKFDSYTRFLKSPLYQECILAEVEGRALPDSQQVPSSPASKHSLGSDHSSVSTPKKLSGKSKSGRSLNEELGDEDSEKKRKGAFFSWSRTRSTGRSQKKREHGDHADDALHANGGLCRRESQGSVSSAGSLDLSEACRTLAPEKDKATKHCCIHLPDGTSCVVAVKAGFSIKDILSGLCERHGINGAAADLFLVGGDKPLVLHQDSSILESRDLRLEKRTLFRLDLVPINRSVGLKAKPTKPVTEVLRPVVARYGLDLSGLLVRLSGEKEPLDLGAPISSLDGQRVVLEEKDPSRGKASADKQKGVPVKQNTAVNSSSRNHSATGEERTLGKSNSIKIKGENGKNARDPRLSKREESIAKIGKKKYQKINLDEAEEFFELISKAQSNRADDQRGLLRKEDLVLPEFLRLPPGSTELTLPTPAAVAKGFSKRSATGNGRESASQPGEQWEPVQESSDSPSTSPGSASSPPGPPGTTPPGQKSPSGPFCTPQSPVSLAQEGTAQIWKRQSQEVEAGGIQTVEDEHVAELTLMGEGDISSPNSTLLPPPSTPQEVPGPSRPGTSRF

SEQ ID NO: 8 - protein, 896 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMNLGKELSNETHVSNDQQSATVSDGELTGADLKDCVSNNSLSSNASLPSVQSCRRLRERRVASWAVSFERLLQDPVGVRYFSDFLRKEFSEENILFWQACEYFNHVPAHDKKELSYRAREIFSKFLCSKATTPVNIDSQAQLADDVLRAPHPDMFKEQQLQIFNLMKFDSYTRFLKSPLYQECILAEVEGRALPDSQQVPSSPASKHSLGSDHSSVSTPKKLSGKSKSGRSLNEELGDEDSEKKRKGAFFSWSRTRSTGRSQKKREHGDHADDALHANGGLCRRESQGSVSSAGSLDLSEACRTLAPEKDKATKHCCIHLPDGTSCVVAVKAGFSIKDILSGLCERHGINGAAADLFLVGGDKPLVLHQDSSILESRDLRLEKRTLFRLDLVPINRSVGLKAKPTKPVTEVLRPVVARYGLDLSGLLVRLSGEKEPLDLGAPISSLDGQRVVLEEKDPSRGKASADKQKGVPVKQNTAVNSSSRNHSATGEERTLGKSNSIKIKGENGKNARDPRLSKREESIAKIGKKKYQKINLDEAEEFFELISKAQSNRADDQRGLLRKEDLVLPEFLRLPPGSTELTLPTPAAVAKGFSKRSATGNGRESASQPGEQWEPVQESSDSPSTSPGSASSPPGPPGTTPPGQKSPSGPFCTPQSPVSLAQEGTAQIWKRQSQEVEAGGIQTVEDEHVAELTLMGEGDISSPNSTLLPPPSTPQEVPGPSRPGSGTHGSRDLPVNRIIDVDLVTGSAPGRDGGIAGAQAGPGRSQASGGPPTSDLPGLGPVPGEPAKPKTSAHHATFV

SEQ ID NO: 9 - protein, 144 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAEAEEFFELISKAQSNRADDQRGLLRKEDLVLPEFLRLPPGSGNGGNA

SEQ ID NO: 10 - protein, 663 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMPGKPKHLGVPNGRMVLAVSDGELSSTTGPQGQGEGRGSSLSIHSLPSGPSSPFPTEEQPVASWALSFERLLQDPLGLAYFTEFLKKEFSAENVTFWKACERFQQIPASDTQQLAQEARNIYQEFLSSQALSPVNIDRQAWLGEEVLAEPRPDMFRAQQLQIFNLMKFDSYARFVKSPLYRECLLAEAEGRPLREPGSSRLGSPDATRKKPKLKPGKSLPLGVEELGQLPPVEGPGGRPLRKSFRRELGGTANAALRRESQGSLNSSASLDLGFLAFVSSKSESHRKSLGSTEGESESRPGKYCCVYLPDGTASLALARPGLTIRDMLAGICEKRGLSLPDIKVYLVGNEQALVLDQDCTVLADQEVRLENRITFELELTALERVVRISAKPTKRLQEALQPILEKHGLSPLEVVLHRPGEKQPLDLGKLVSSVAAQRLVLDTLPGVKISKARDKSPCRSQGCPPRTQDKATHPPPASPSSLVKVPSSATGKRQTCDIEGLVELLNRVQSSGAHDQRGLLRKEDLVLPEFLQLPAQGPSSEETPPQTKSAAQPIGGSLNSTTDSAL

SEQ ID NO: 11 - protein, 142 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNATGKRQTCDIEGLVELLNRVQSSGAHDQRGLLRKEDLVLPEFLQLP

SEQ ID NO: 12 - protein, 308 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMQSAMFLAVQHDCRPMDKSAGSGHKSEEKREKMKRTLLKDWKTRLSYFLQNSSTPGKPKTGKKSKQQAFIKPSPEEAQLWSEAFDELLASKYGLAAFRAFLKSEFCEENIEFWLACEDFKKTKSPQKLSSKARKIYTDFIEKEAPKEINIDFQTKTLIAQNIQEATSGCFTTAQKRVYSLMENNSYPRFLESEFYQDLCKKPQITTEPHAT

SEQ ID NO: 13 - protein, 1014 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMNRFNGLCKVCSERRYRQITIPRGKDGFGFTICCDSPVRVQAVDSGGPAERAGLQQLDTVLQLNERPVEHWKCVELAHEIRSCPSEIILLVWRMVPQVKPGPDGGVLRRASCKSTHDLQSPPNKREKNCTHGVQARPEQRHSCHLVCDSSDGLLLGGWERYTEVAKRGGQHTLPALSRATAPTDPNYIILAPLNPGSQLLRPVYQEDTIPEESGSPSKGKSYTGLGKKSRLMKTVQTMKGHGNYQNCPVVRPHATHSSYGTYVTLAPKVLVFPVFVQPLDLCNPARTLLLSEELLLYEGRNKAAEVTLFAYSDLLLFTKEDEPGRCDVLRNPLYLQSVKLQEGSSEDLKFCVLYLAEKAECLFTLEAHSQEQKKRVCWCLSENIAKQQQLAASPPDSKMFETEADEKREMALEEGKGPGAEDSPPSKEPSPGQELPPGQDLPPNKDSPSGQEPAPSQEPLSSKDSATSEGSPPGPDAPPSKDVPPCQEPPPAQDLSPCQDLPAGQEPLPHQDPLLTKDLPAIQESPTRDLPPCQDLPPSQVSLPAKALTEDTMSSGDLLAATGDPPAAPRPAFVIPEVRLDSTYSQKAGAEQGCSGDEEDAEEAEEVEEGEEGEEDEDEDTSDDNYGERSEAKRSSMIETGQGAEGGLSLRVQNSLRRRTHSEGSLLQEPRGPCFASDTTLHCSDGEGAASTWGMPSPSTLKKELGRNGGSMHHLSLFFTGHRKMSGADTVGDDDEASRKRKSKNLAKDMKNKLGIFRRRNESPGAPPAGKADKMMKSFKPTSEEALKWGESLEKLLVHKYGLAVFQAFLRTEFSEENLEFWLACEDFKKVKSQSKMASKAKKIFAEYIAIQACKEVNLDSYTREHTKDNLQSVTRGCFDLAQKRIFGLMEKDSYPRFLRSDLYLDLINQKKMSPPL

SEQ ID NO: 14 - protein, 616 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMFETEADEKREMALEEGKGPGAEDSPPSKEPSPGQELPPGQDLPPNKDSPSGQEPAPSQEPLSSKDSATSEGSPPGPDAPPSKDVPPCQEPPPAQDLSPCQDLPAGQEPLPHQDPLLTKDLPAIQESPTRDLPPCQDLPPSQVSLPAKALTEDTMSSGDLLAATGDPPAAPRPAFVIPEVRLDSTYSQKAGAEQGCSGDEEDAEEAEEVEEGEEGEEDEDEDTSDDNYGERSEAKRSSMIETGQGAEGGLSLRVQNSLRRRTHSEGSLLQEPRGPCFASDTTLHCSDGEGAASTWGMPSPSTLKKELGRNGGSMHHLSLFFTGHRKMSGADTVGDDDEASRKRKSKNLAKDMKNKLGIFRRRNESPGAPPAGKADKMMKSFKPTSEEALKWGESLEKLLVHKYGLAVFQAFLRTEFSEENLEFWLACEDFKKVKSQSKMASKAKKIFAEYIAIQACKEVNLDSYTREHTKDNLQSVTRGCFDLAQKRIFGLMEKDSYPRFLRSDLYLDLINQKKMSPPL

SEQ ID NO: 15 - protein, 265 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMKNKLGIFRRRNESPGAPPAGKADKMMKSFKPTSEEALKWGESLEKLLVHKYGLAVFQAFLRTEFSEENLEFWLACEDFKKVKSQSKMASKAKKIFAEYIAIQACKEVNLDSYTREHTKDNLQSVTRGCFDLAQKRIFGLMEKDSYPRFLRSDLYLDLINQKKMSPPL

SEQ ID NO: 16 - protein, 408 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMVTRRPVTNSWDWLPAGAAPEAVPCRHMPLSRLPLRVGQKEFFFPLPLLVPPISWLLLSESQPRLVPGSPVIRPGFQRACVAAACTVAARCPGRGVGDRSQSGASYRPICGPKVGGPTEMLRGMYLTRNGNLQRRHTMKEAKDMKNKLGIFRRRNESPGAPPAGKADKMMKSFKPTSEEALKWGESLEKLLVHKYGLAVFQAFLRTEFSEENLEFWLACEDFKKVKSQSKMASKAKKIFAEYIAIQACKEVNLDSYTREHTKDNLQSVTRGCFDLAQKRIFGLMEKDSYPRFLRSDLYLDLINQKKMSPPL

SEQ ID NO: 17 - protein, 1295 aa, Artificial Sequence [sequence omitted]

SEQ ID NO: 18 - protein, 399 aa, Artificial Sequence [sequence omitted]

SEQ ID NO: 19 - protein, 302 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMCKGLAGLPASCLRSAKDMKHRLGFLLQKSDSCEHNSSHNKKDKVVICQRVSQEEVKKWAESLENLISHECGLAAFKAFLKSEYSEENIDFWISCEEYKKIKSPSKLSPKAKKIYNEFISVQATKEVNLDSCTREETSRNMLEPTITCFDEAQKKIFNLMEKDSYRRFLKSRFYLDLVNPSSCGAEKQKGAKSSADCASLVPQCA

SEQ ID NO: 20 - protein, 1054 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMLTEASLSIWGWGSLGIVLFLITFGPFVIFYLTFYILCFVGGGLVVTLLFGKTNSEKYLEQCEHSFLPPTSPGVPKCLEEMKREARTIKIDRRLTGANIIDEPLQQVIQFSLRDYVQYWYYTLSDDESFLLEIRQTLQNALIQFATRSKEIDWQPYFTTRIVDDFGTHLRVFRKAQQKITEKDDQVKGTAEDLVDTFFEVEVEMEKEVCRDLVCTSPKDEEGFLRDLCEVLLYLLLPPGDFQNKIMRYFVREILARGILLPLINQLSDPDYINQYVIWMIRDSNCNYEAFMNIIKLSDNIGELEAVRDKAAEELQYLRSLDTAGDDINTIKNQINSLLFVKKVCDSRIQRLQSGKEINTVKLAANFGKLCTVPLDSILVDNVALQFFMDYMQQTGGQAHLFFWMTVEGYRVTAQQQLEVLLSRQRDGKHQTNQTKGLLRAAAVGIYEQYLSEKASPRVTVDDYLVAKLADTLNHEDPTPEIFDDIQRKVYELMLRDERFYPSFRQNALYVRMLAELDMLKDPSFRGSDDGDGESFNGSPTGSINLSLDDLSNVSSDDSVQLHAYISDTGVCNDHGKTYALYAITVHRRNLNSEEMWKTYRRYSDFHDFHMRITEQFESLSSILKLPGKKTFNNMDRDFLEKRKKDLNAYLQLLLAPEMMKASPALAHYVYDFLENKAYSKGKGDFARKMDTFVNPLRNSMRNVSNAVKSLPDSLAEGMTKMSDNMGKMSERLGQDIKQSFFKVPPLIPKTDSDPEHRRVSAQLDDNVDDNIPLRVMLLLMDEVFDLKERNQWLRRNIKNLLQQLIRATYGDTINRKIVDHVDWMTSPEQVADSVKRFRDAFWPNGILAEAVPCRDKSIRMRTRVAGKTKLLAIMPDELKHIIGAETTRKGILRVFEMFQHNQLNRRMVYVFLEGFLETLFPQYKFRELFNKLHSRSKQMQKYKQKLQTTQAPSLQKR

SEQ ID NO: 21 - protein, 1347 aa, Artificial Sequence
  Synthetic peptide
MGCTLSAEDKAAVERSKMIDRNLREDGEKAAREVKLLLLGAGESGKSTIVKQMKIIHEAGYSEEECKQYKAVVYSNTIQSIIAIIRAMGRLGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAKIDFGDSARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVAAIIFCVALSDYDLVLAEDEEMNRMHESMKLFDSICNNKWFTDTSIILFLNKKDLFEEKIKKSPLTICYQEYAGSNTYEEAAAYIQCQFEDLNKRKDTKEIYTHFTCATDTKNVQFVFDAVTDVIIKNNLKDCGLF

SEQ ID NO: 22 - protein, 1347 aa, Artificial Sequence
  Synthetic peptide
MGCTVSAEDKAAAERSKMIDKNLREDGEKAAREVKLLLLGAGESGKSTIVKQMKIIHEDGYSEEECRQYRAVVYSNTIQSIMAIVKAMGNLGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAQIDFADPSRADDARQLFALSCTAEEQGVLPDDLSGVIRRLWADHGVQACFGRSREYQLNDSAAYYLNDLERIAQSDYIPTQQDVLRTRVKTTGIVETHFTFKDLHFKMFDVGGQRSERKKWIHCFEGVTAIIFCVALSAYDLVLAEDEEMNRMHESMKLFDSICNNKWFTDTSIILFLNKKDLFEEKITHSPLTICFPEYTGANKYDEAASYIQSKFEDLNKRKDTKEIYTHFTCATDTKNVQFVFDAVTDVIIKNNLKDCGL

SEQ ID NO: 23 - protein, 1347 aa, Artificial Sequence
  Synthetic peptide
MGCTLSAEDKAAVERSKMIDRNLREDGEKAAKEVKLLLLGAGESGKSTIVKQMKIIHEDGYSEDECKQYKVVVYSNTIQSIIAIIRAMGRLGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAKIDFGEAARADDARQLFVLAGSAEEGVMTPELAGVIKRLWRDGGVQACFSRSREYQLNDSASYYLNDLDRISQSNYIPTQQDVLRTRVKTTGIVETHFTFKDLYFKMFDVGGQRSERKKWIHCFEGVTAIIFCVALSDYDLVLAEDEEMNRMHESMKLFDSICNNKWFTETSIILFLNKKDLFEEKIKRSPLTICYPEYTGSNTYEEAAAYIQCQFEDLNRRKDTKEIYTHFTCATDTKNVQFVFDAVTDVIIKNNLKECGLY

SEQ ID NO: 24 - protein, 1343 aa, Artificial Sequence
  Synthetic peptide
MGAGASAEEKHSRELEKKLKEDAEKDARTVKLLLLGAGESGKSTIVKQMKIIHQDGYSLEECLEFIAIIYGNTLQSILAIVRAMTTLGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNANIQYGDSARQDDARKLMHMADTIEEGTMPKEMSDIIQRLWKDSGIQACFERASEYQLNDSAGYYLSDLERLVTPGYVPTEQDVLRSRVKTTGIIETQFSFKDLNFRMFDVGGQRSERKKWIHCFEGVTCIIFIAALSAYDMVLVEDDEVNRMHESLHLFNSICNHRYFATTSIVLFLNKKDVFFEKIKKAHLSICFPDYDGPNTYEDAGNYIKVQFLELNMRRDVKEIYSHMTCATDTQNVKFVFDAVTDIIIKENLKDCGLF

SEQ ID NO: 25 - protein, 1347 aa, Artificial Sequence
  Synthetic peptide
MGSGASAEDKELAKRSKELEKKLQEDADKEAKTVKLLLLGAGESGKSTIVKQMKIIHQDGYSPEECLEFKAIIYGNVLQSILAIIRAMTTLGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAGIDYAEPSCADDGRQLNNLADSIEEGTMPPELVEVIRRLWKDGGVQACFERAAEYQLNDSASYYLNQLERITDPEYLPSEQDVLRSRVKTTGIIETKFSVKDLNFRMFDVGGQRSERKKWIHCFEGVTCIIFCAALSAYDMVLVEDDEVNRMHESLHLFNSICNHKFFAATSIVLFLNKKDLFEEKIKKVHLSICFPEYDGNNSYDDAGNYIKSQFLDLNMRKDVKEIYSHMTCATDTQNVKFVFDAVTDIIIKENLKDCGLF

SEQ ID NO: 26 - protein, 1347 aa, Artificial Sequence
  Synthetic peptide
MGSGISSESKESAKRSKELEKKLQEDAERDARTVKLLLLGAGESGKSTIVKQMKIIHKNGYSEQECMEFKAVIYSNTLQSILAIVKAMTTLGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAGIDYVNPRSAEDQRQLYAMANTLEDGGMTPQLAEVIKRLWRDPGIQACFERASEYQLNDSAAYYLNDLDRITASGYVPNEQDVLHSRVKTTGIIETQFSFKDLHFRMFDVGGQRSERKKWIHCFEGVTCIIFCAALSAYDMVLVEDEEVNRMHESLHLFNSICNHKYFSTTSIVLFLNKKDIFQEKVTKVHLSICFPEYTGPNTFEDAGNYIKNQFLDLNLKKEDKEIYSHMTCATDTQNVKFVFDAVTDIIIKENLKDCGLF

SEQ ID NO: 27 - protein, 1352 aa, Artificial Sequence
  Synthetic peptide
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 28 - protein, 1367 aa, Artificial Sequence [sequence omitted]

SEQ ID NO: 29 - protein, 1352 aa, Artificial Sequence
  Synthetic peptide
MTLESMMACCLSDEVKESKRINAEIEKQLRRDKRDARRELKLLLLGTGESGKSTFIKQMRIIHGAGYSEEDKRGFTKLVYQNIFTAMQAMIRAMETLKILYKYEQNKANALLIREVDVEKVTTFGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAEHQYVSAIKTLWEDPGIQECYDRRREYQLSDSAKYYLTDVDRIATLGYLPTQQDVLRVRVPTTGIIEYPFDLENIIFRMVDVGGQRSERRKWIHCFENVTSIMFLVALSEYDQVLVESDNENRMEESKALFRTIITYPWFQNSSVILFLNKKDLLEDKILYSHLVDYFPEFDGPQRDAQAAREFILKMFVDLNPDSDKIIYSHFTCATDTENIRFVFAAVKDTILQLNLKEYNLV

SEQ ID NO: 30 - protein, 1382 aa, Artificial Sequence
  Synthetic peptide
MGCLGNSKTEDQRNEEKAQREANKKIEKQLQKDKQVYRATHRLLLLGAGESGKSTIVKQMRILHVNGFNGEGGEEDGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNASNSDGEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 31 - protein, 1373 aa, Artificial Sequence
  Synthetic peptide
MGCLGNSKTEDQRNEEKAQREANKKIEKQLQKDKQVYRATHRLLLLGAGESGKSTIVKQMRILHVNGFNGDGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNASEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 32 - protein, 1383 aa, Artificial Sequence
  Synthetic peptide
MGCLGNSKTEDQRNEEKAQREANKKIEKQLQKDKQVYRATHRLLLLGAGESGKSTIVKQMRILHVNGFNGEGGEEDGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNASNSDGSEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 33 - protein, 1372 aa, Artificial Sequence
  Synthetic peptide
MGCLGNSKTEDQRNEEKAQREANKKIEKQLQKDKQVYRATHRLLLLGAGESGKSTIVKQMRILHVNGFNGDGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAEKATKVQDIKNNLKEAIETIVAAMSNLVPPVELANPENQFRVDYILSVMNVPDFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 34 - protein, 1451 aa, Artificial Sequence
  Synthetic peptide
MGLCYSLRPLLFGGPGDDPCAASEPPVEDAQPAPAPALAPVRAAARDTARTLLPRGGEGSPACARPKADKPKEKRQRTEQLSAEEREAAKEREAVKEARKVSRGIDRMLRDQKRDLQQTHRLLLLGAGESGKSTIVKQMRILHVNGFNPEGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAEKKQKILDIRKNVKDAIVTIVSAMSTIIPPVPLANPENQFRSDYIKSIAPITDFEYSQEFFDHVKKLWDDEGVKACFERSNEYQLIDCAQYFLERIDSVSLVDYTPTDQDLLRCRVLTSGIFETRFQVDKVNFHMFDVGGQRDERRKWIQCFNDVTAIIYVAACSSYNMVIREDNNTNRLRESLDLFESIWNNRWLRTISIILFLNKQDMLAEKVLAGKSKIEDYFPEYANYTVPEDATPDAGEDPKVTRAKFFIRDLFLRISTATGDGKHYCYPHFTCAVDTENIRRVFNDCRDIIQRMHLKQYELL

SEQ ID NO: 35 - protein, 1374 aa, Artificial Sequence
  Synthetic peptide
MGCLGGNSKTTEDQGVDEKERREANKKIEKQLQKERLAYKATHRLLLLGAGESGKSTIVKQMRILHVNGFNPEGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQKGNGGNAEKKQKILDIRKNVKDAIVTIVSAMSTIIPPVPLANPENQFRSDYIKSIAPITDFEYSQEFFDHVKKLWDDEGVKACFERSNEYQLIDCAQYFLERIDSVSLVDYTPTDQDLLRCRVLTSGIFETRFQVDKVNFHMFDVGGQRDERRKWIQCFNDVTAIIYVAACSSYNMVIREDNNTNRLRESLDLFESIWNNRWLRTISIILFLNKQDMLAEKVLAGKSKIEDYFPEYANYTVPEDATPDAGEDPKVTRAKFFIRDLFLRISTATGDGKHYCYPHFTCAVDTENIRRVFNDCRDIIQRMHLKQYELL

SEQ ID NO: 36 - DNA, 5424 bp, Artificial Sequence
  Synthetic oligonucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccgcggccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 37 - DNA, 10059 bp, Artificial Sequence
  Synthetic oligonucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgggaaacgggggaaacgcaatgtttagagctggggaggcctccaaacgcccattgcctgggccgtcgcccccaagggtgcggagtgtggaggttgcccgggggagggccggctacggattcacgctttcgggacaggcaccctgtgtgctcagctgcgtcatgagagggagccctgcggatttcgtgggcctccgagctggagaccagatacttgctgtcaatgaaatcaacgtgaaaaaagcatctcatgaagatgtagtgaaattaattgggaagtgctctggtgtccttcacatggtgattgctgaaggcgtcggccgcttcgaatcctgttccagtgatgaagaagggggactctatgaaggaaaaggctggctgaagcccaagctggattctaaagcactaggtataaacagagcagagcgagtcgtggaggaaatgcagtctggtggaattttcaatatgatttttgaaaacccgagcctttgtgcgagcaattcagagcccttgaaattgaaacaaagatccctttcagagtcggccgcaactcgatttgatgttggacatgaaagtataaataatccaaatcccaacatgctttctaaggaggaaatatcaaaagttattcatgatgattcggttttcagcattggactagaaagtcatgacgattttgcattggatgcaagtattttaaacgtggcgatgatcgtgggctacttaggctccattgagcttccttccacgagctccaacctggagtccgacagcttgcaagccatccgcggctgcatgcggcgcctgcgggcagagcagaaaatccactcgctggtgaccatgaagatcatgcacgactgtgtgcagctgagcactgacaaggctggagtcgtggccgagtacccggccgagaagctggccttcagcgccgtgtgcccggacgaccggcgatttttcgggttggttaccatgcagacgaatgacgacgggagcctggcccaggaggaggagggcgccctgcggacttcctgccacgtgttcatggtggacccagacttgtttaatcacaagatccaccaaggcattgctcggcggtttgggtttgagtgcacggccgacccagacaccaatggctgtctggaattcccggcgtcctccctccccgtcctgcagttcatctctgtcctgtaccgagacatgggtgagctgattgagggcatgcgggcccgcgcctttctggacggggacgccgatgcccaccagaacaacagcaccagcagcaacagtgacagcggcattgggaacttccaccaggaggagaagagcaaccgggtccttgtggtggacctgggtgggagctcgagcagacacggccccggaggcagcgcgtgggacggtgtgggtgggaggggtgcccagccctggggtgctccctggactgggcccttctgtccggaccccgaagggagccccccatttgaggccgctcatcagactgacaggttctgggacctaaacaagcacctagggccagcctctcctgtggaggtgcccccagcttccttgaggagctcagtccccccttccaagaggggcaccgtgggtgctggctgtggtttcaaccagcgctggctcccggtccacgtgctccgggagtggcagtgcggacacaccagcgaccaggactcttacacagattccaccgatggctggtccagcatcaactgcggcacactgccccctcctatgagcaagatccccgcagaccgctacagggtggagggcagcttcgcgcagcccccgctgaatgccccgaagagggagtggtccaggaaggcctttggaatgcaaagcatttttggtccccatcgaaatgttcgaaagactaaggaagataaaaagggctcaaaatttgggcggggaactggactcactcagccttctcaacgcacgtctgctcggagatcatttgggagatccaagagattcagtatcactcgctcccttgatgatcttgagtctgcaactgtgtctgatggcgagttgacgggcgccgacctgaaggactgcgtcagcaacaacagcctgagcagcaatgccagcctccccagcgtgcagagctgccggcgcctgcgtgagaggagggtcgccagctgggccgtgtcctttgagcgcctgctgcaggaccccgtcggtgtccgctacttctctgattttctaaggaaagaattcagtgaagaaaacattttattctggcaggcctgtgaatattttaatcatgttcctgcacatgacaaaaaggagctttcctacagggcccgggagattttcagtaagtttctctgcagcaaagccaccaccccggtcaacatcgacagccaggcccagctagcagacgacgtcctccgcgcacctcacccagacatgttcaaggagcagcagctgcagatcttcaatctcatgaagtttgatagctacactcgctttctgaagtccccgctgtaccaggaatgcatcctggcggaagtggagggccgtgcactcccggactcgcagcaggtccccagcagcccggcttccaagcacagcctcggttcagaccactccagtgtgtccacgccaaaaaagttaagtggaaaatcaaaatccggccgatccctgaatgaagagctgggggatgaggacagcgagaagaagcggaaaggcgcgtttttctcgtggtcgcggaccaggagcaccgggaggtcccagaaaaagagggagcacggggaccacgcagacgacgccctgcatgccaatggaggcctgtgtcgccgagagtcgcagggctctgtgtcctctgcggggagcctggacctgtcggaggcctgcaggactttggcacccgagaaggacaaggccaccaagcactgctgcattcatctcccggatgggacatcctgcgtggtggctgtcaaggcgggcttctccatcaaagacatcctgtccggactctgtgagcggcatggcatcaacggggcggccgcggacctcttcctggtgggcggggacaagcctctggtgctgcaccaagacagtagcatcttggagtcaagggacctgcgcctagaaaagcgcaccttgtttcggctggatcttgttccgattaaccggtcagtgggactcaaggccaagcccaccaagcccgtcacggaggtgctgcggcccgtggtggccagatacggcctggacctcagtggcctgctggtgaggctgagtggagagaaggagcccctggaccttggcgcccctatatcgagtctggacggacagcgggttgtcttggaggagaaggatccttccagaggaaaggcatccgcagataaacagaaaggtgtgccagtgaaacagaacacagctgtaaattccagctccagaaaccactcggctacgggagaggaaagaacactaggcaagtctaattctattaaaataaaaggagaaaatggaaaaaatgctagggatccccggctttcaaagagagaagaatctattgcaaagattgggaaaaaaaaatatcagaaaattaatttggacgaagcagaggagttttttgagcttatttccaaagctcagagcaacagagcagatgaccaacgtgggctgctaaggaaggaagacctggtgttgccagagttcctccgtttacctcctggttccacagaactcaccctccccactccagctgctgtggccaagggctttagcaagagaagcgccacaggcaacggccgggagagcgcctcccagcctggcgagcagtgggagccagtccaggagagcagcgacagcccgtccaccagcccgggctcagcctccagcccccctggacctcctgggacgaccccccccgggcagaagtctcccagcgggcccttctgcactccccagtcccccgtctccctcgcgcaggagggcaccgcccagatctggaagaggcagtctcaggaagtggaggccgggggcatccagacggtggaggatgagcacgtggccgagctgaccctgatgggggagggggacatcagcagccccaacagcaccttgctgccgccgccctccaccccccaggaagtgccaggaccttccagaccaggaagtgggacccatggcagccgagacctcccagtcaacagaatcatcgatgtggatcttgtaactggctcggcgcccgggcgggatggtggcatagcgggggcacaggctggccctgggaggtcgcaggccagtggtgggcctcctacatcagacctccctggcttgggccccgtcccgggtgagcctgctaagcccaagaccagcgctcaccacgccaccttcgtctgagcggccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 38 - DNA, 9468 bp, Artificial Sequence
  Synthetic oligonucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgggctgcacgctgagcgccgaggacaaggcggcggtggagcggagtaagatgatcgaccgcaacctccgtgaggacggcgagaaggcggcgcgcgaggtcaagctgctgctgctcggtgctggtgaatctggtaaaagtacaattgtgaagcagatgaaaattatccatgaagctggttattcagaagaggagtgtaaacaatacaaagcagtggtctacagtaacaccatccagtcaattattgctatcattagggctatggggaggttgggaaacgggggaaacgcacgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaaggaaacgggggaaacgcaaagatagactttggtgactcagcccgggcggatgatgcacgccaactctttgtgctagctggagctgctgaagaaggctttatgactgcagaacttgctggagttataaagagattgtggaaagatagtggtgtacaagcctgtttcaacagatcccgagagtaccagcttaatgattctgcagcatactatttgaatgacttggacagaatagctcaaccaaattacatcccgactcaacaagatgttctcagaactagagtgaaaactacaggaattgttgaaacccattttactttcaaagatcttcattttaaaatgtttgatgtgggaggtcagagatctgagcggaagaagtggattcattgcttcgaaggagtggcggcgatcatcttctgtgtagcactgagtgactacgacctggttctagctgaagatgaagaaatgaaccgaatgcatgaaagcatgaaattgtttgacagcatatgtaacaacaagtggtttacagatacatccattatactttttctaaacaagaaggatctttttgaagaaaaaatcaaaaagagccctctcactatatgctatcaagaatatgcaggatcaaacacatatgaagaggcagctgcatatattcaatgtcagtttgaagacctcaataaaagaaaggacacaaaggaaatatacacccacttcacatgtgccacagatactaagaatgtgcagtttgtttttgatgctgtaacagatgtcatcataaaaaataatctaaaagattgtggtctcttttaagcggccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 39 - DNA, 6092 bp, Artificial Sequence
  Synthetic oligionucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 40 - DNA, 14771 bp, Artificial Sequence
  Synthetic oligonucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgggaaacgggggaaacgcaatgtttagagctggggaggcctccaaacgcccattgcctgggccgtcgcccccaagggtgcggagtgtggaggttgcccgggggagggccggctacggattcacgctttcgggacaggcaccctgtgtgctcagctgcgtcatgagagggagccctgcggatttcgtgggcctccgagctggagaccagatacttgctgtcaatgaaatcaacgtgaaaaaagcatctcatgaagatgtagtgaaattaattgggaagtgctctggtgtccttcacatggtgattgctgaaggcgtcggccgcttcgaatcctgttccagtgatgaagaagggggactctatgaaggaaaaggctggctgaagcccaagctggattctaaagcactaggtataaacagagcagagcgagtcgtggaggaaatgcagtctggtggaattttcaatatgatttttgaaaacccgagcctttgtgcgagcaattcagagcccttgaaattgaaacaaagatccctttcagagtcggccgcaactcgatttgatgttggacatgaaagtataaataatccaaatcccaacatgctttctaaggaggaaatatcaaaagttattcatgatgattcggttttcagcattggactagaaagtcatgacgattttgcattggatgcaagtattttaaacgtggcgatgatcgtgggctacttaggctccattgagcttccttccacgagctccaacctggagtccgacagcttgcaagccatccgcggctgcatgcggcgcctgcgggcagagcagaaaatccactcgctggtgaccatgaagatcatgcacgactgtgtgcagctgagcactgacaaggctggagtcgtggccgagtacccggccgagaagctggccttcagcgccgtgtgcccggacgaccggcgatttttcgggttggttaccatgcagacgaatgacgacgggagcctggcccaggaggaggagggcgccctgcggacttcctgccacgtgttcatggtggacccagacttgtttaatcacaagatccaccaaggcattgctcggcggtttgggtttgagtgcacggccgacccagacaccaatggctgtctggaattcccggcgtcctccctccccgtcctgcagttcatctctgtcctgtaccgagacatgggtgagctgattgagggcatgcgggcccgcgcctttctggacggggacgccgatgcccaccagaacaacagcaccagcagcaacagtgacagcggcattgggaacttccaccaggaggagaagagcaaccgggtccttgtggtggacctgggtgggagctcgagcagacacggccccggaggcagcgcgtgggacggtgtgggtgggaggggtgcccagccctggggtgctccctggactgggcccttctgtccggaccccgaagggagccccccatttgaggccgctcatcagactgacaggttctgggacctaaacaagcacctagggccagcctctcctgtggaggtgcccccagcttccttgaggagctcagtccccccttccaagaggggcaccgtgggtgctggctgtggtttcaaccagcgctggctcccggtccacgtgctccgggagtggcagtgcggacacaccagcgaccaggactcttacacagattccaccgatggctggtccagcatcaactgcggcacactgccccctcctatgagcaagatccccgcagaccgctacagggtggagggcagcttcgcgcagcccccgctgaatgccccgaagagggagtggtccaggaaggcctttggaatgcaaagcatttttggtccccatcgaaatgttcgaaagactaaggaagataaaaagggctcaaaatttgggcggggaactggactcactcagccttctcaacgcacgtctgctcggagatcatttgggagatccaagagattcagtatcactcgctcccttgatgatcttgagtctgcaactgtgtctgatggcgagttgacgggcgccgacctgaaggactgcgtcagcaacaacagcctgagcagcaatgccagcctccccagcgtgcagagctgccggcgcctgcgtgagaggagggtcgccagctgggccgtgtcctttgagcgcctgctgcaggaccccgtcggtgtccgctacttctctgattttctaaggaaagaattcagtgaagaaaacattttattctggcaggcctgtgaatattttaatcatgttcctgcacatgacaaaaaggagctttcctacagggcccgggagattttcagtaagtttctctgcagcaaagccaccaccccggtcaacatcgacagccaggcccagctagcagacgacgtcctccgcgcacctcacccagacatgttcaaggagcagcagctgcagatcttcaatctcatgaagtttgatagctacactcgctttctgaagtccccgctgtaccaggaatgcatcctggcggaagtggagggccgtgcactcccggactcgcagcaggtccccagcagcccggcttccaagcacagcctcggttcagaccactccagtgtgtccacgccaaaaaagttaagtggaaaatcaaaatccggccgatccctgaatgaagagctgggggatgaggacagcgagaagaagcggaaaggcgcgtttttctcgtggtcgcggaccaggagcaccgggaggtcccagaaaaagagggagcacggggaccacgcagacgacgccctgcatgccaatggaggcctgtgtcgccgagagtcgcagggctctgtgtcctctgcggggagcctggacctgtcggaggcctgcaggactttggcacccgagaaggacaaggccaccaagcactgctgcattcatctcccggatgggacatcctgcgtggtggctgtcaaggcgggcttctccatcaaagacatcctgtccggactctgtgagcggcatggcatcaacggggcggccgcggacctcttcctggtgggcggggacaagcctctggtgctgcaccaagacagtagcatcttggagtcaagggacctgcgcctagaaaagcgcaccttgtttcggctggatcttgttccgattaaccggtcagtgggactcaaggccaagcccaccaagcccgtcacggaggtgctgcggcccgtggtggccagatacggcctggacctcagtggcctgctggtgaggctgagtggagagaaggagcccctggaccttggcgcccctatatcgagtctggacggacagcgggttgtcttggaggagaaggatccttccagaggaaaggcatccgcagataaacagaaaggtgtgccagtgaaacagaacacagctgtaaattccagctccagaaaccactcggctacgggagaggaaagaacactaggcaagtctaattctattaaaataaaaggagaaaatggaaaaaatgctagggatccccggctttcaaagagagaagaatctattgcaaagattgggaaaaaaaaatatcagaaaattaatttggacgaagcagaggagttttttgagcttatttccaaagctcagagcaacagagcagatgaccaacgtgggctgctaaggaaggaagacctggtgttgccagagttcctccgtttacctcctggttccacagaactcaccctccccactccagctgctgtggccaagggctttagcaagagaagcgccacaggcaacggccgggagagcgcctcccagcctggcgagcagtgggagccagtccaggagagcagcgacagcccgtccaccagcccgggctcagcctccagcccccctggacctcctgggacgaccccccccgggcagaagtctcccagcgggcccttctgcactccccagtcccccgtctccctcgcgcaggagggcaccgcccagatctggaagaggcagtctcaggaagtggaggccgggggcatccagacggtggaggatgagcacgtggccgagctgaccctgatgggggagggggacatcagcagccccaacagcaccttgctgccgccgccctccaccccccaggaagtgccaggaccttccagaccaggaagtgggacccatggcagccgagacctcccagtcaacagaatcatcgatgtggatcttgtaactggctcggcgcccgggcgggatggtggcatagcgggggcacaggctggccctgggaggtcgcaggccagtggtgggcctcctacatcagacctccctggcttgggccccgtcccgggtgagcctgctaagcccaagaccagcgctcaccacgccaccttcgtctgaacgcgtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatccatgggctgcacgctgagcgccgaggacaaggcggcggtggagcggagtaagatgatcgaccgcaacctccgtgaggacggcgagaaggcggcgcgcgaggtcaagctgctgctgctcggtgctggtgaatctggtaaaagtacaattgtgaagcagatgaaaattatccatgaagctggttattcagaagaggagtgtaaacaatacaaagcagtggtctacagtaacaccatccagtcaattattgctatcattagggctatggggaggttgggaaacgggggaaacgcacgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaaggaaacgggggaaacgcaaagatagactttggtgactcagcccgggcggatgatgcacgccaactctttgtgctagctggagctgctgaagaaggctttatgactgcagaacttgctggagttataaagagattgtggaaagatagtggtgtacaagcctgtttcaacagatcccgagagtaccagcttaatgattctgcagcatactatttgaatgacttggacagaatagctcaaccaaattacatcccgactcaacaagatgttctcagaactagagtgaaaactacaggaattgttgaaacccattttactttcaaagatcttcattttaaaatgtttgatgtgggaggtcagagatctgagcggaagaagtggattcattgcttcgaaggagtggcggcgatcatcttctgtgtagcactgagtgactacgacctggttctagctgaagatgaagaaatgaaccgaatgcatgaaagcatgaaattgtttgacagcatatgtaacaacaagtggtttacagatacatccattatactttttctaaacaagaaggatctttttgaagaaaaaatcaaaaagagccctctcactatatgctatcaagaatatgcaggatcaaacacatatgaagaggcagctgcatatattcaatgtcagtttgaagacctcaataaaagaaaggacacaaaggaaatatacacccacttcacatgtgccacagatactaagaatgtgcagtttgtttttgatgctgtaacagatgtcatcataaaaaataatctaaaagattgtggtctcttttaatctagagtcgacccgggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagagctttacggcacctcgaccgcaaaaaacttgatttgggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaatatttaacgcgaattttaacaaaatattaacgtttacaatttcgcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 41 - protein, 6 aa, Artificial Sequence
  Synthetic peptide
GNGGNA

SEQ ID NO: 42 - DNA, 18 bp, Artificial Sequence
  Synthetic oligonucleotide
ggaaacgggggaaacgca